(19) United States
(12) Patent Application Publication
(10) Pub. No.: US 2023/0218548 A1
(43) Pub. Date: Jul. 13, 2023

(54) NITRO-AMINOADAMANTANE COMPOUNDS FOR THE TREATMENT OF BETACORONAVIRUS INFECTIONS

(71) Applicant: EuMentis Therapeutics, Inc., Newton, MA (US)

(72) Inventors: Mark Allan TEPPER, Newton, MA (US); Stuart Arthur LIPTON, Rancho Santa Fe, CA (US)

(21) Appl. No.: 17/912,611

(22) PCT Filed: Mar. 19, 2021
(86) PCT No.: PCT/US2021/023092

§ 371 Date: Sep. 19, 2022

Related U.S. Application Data
(60) Provisional application No. 62/991,851, filed on Mar. 19, 2020.

(51) Int. Cl. A61K 31/13; A61P 31/14

(52) U.S. Cl. CPC A61K 31/13; A61P 31/14

(57) ABSTRACT

The invention features the use of nitro-aminoadamantane compounds for the treatment and prophylaxis of severe symptoms of beta-coronavirus infections, such as infections by SARS-CoV-2, SARS-CoV-1, MERS-CoV, and related viruses.

### BACKGROUND OF THE INVENTION

During the first weeks of 2020, the world has evidenced the emergence of a new human pathogen that achieved enough zoonotic spillover to cause a pandemic, from a highly pathogenic betacoronavirus.

The 2019 novel Coronavirus (SARS-CoV-2) that is the cause of the highly infectious disease known as COVID-19, is a new member of a group, that includes previously recognized zoonotic pathogens, as is the case of the Severe Acute Respiratory Syndrome Coronavirus (SARS-CoV-1), that caused epidemics in China in 2002-2003, and the Middle East Respiratory Syndrome (MERS-CoV), affecting Saudi Arabia and neighbor countries in 2012-2013.

Based on hospitalized patient data, the majority of COVID-19 cases (about 80%) present with asymptomatic or mild symptoms while the remainder are severe or critical (Huang et al., Lancet 395:497 (2020); Chan et al., Lancet 395:514 (2020)). It seems that the severity and fatality rate of COVID-19 is milder than that of SARS and MERS, but infectivity is greater. With similar clinical presentations as SARS and MERS, the most common symptoms of COVID-19 are fever, fatigue, and respiratory symptoms, including cough, sore throat and shortness of breath. A study of 41 hospitalized patients showed high-levels of proinflammatory cytokines were observed in the COVID-19 severe cases (Huang et al., Lancet 395:497 (2020). These findings are in line with SARS and MERS in that the presence of lymphopenia and “cytokine storm” likely plays a major role in the pathogenesis of COVID-19 (see, e.g., Nicholls et al., Lancet.; 381(9371):1773 (2003); Mahallawi et al., Cytokine.; 104:8 (2018); and Wong et al., Clin Exp Immunol. 138(1):95 (2004)). This so-called “cytokine storm” can initiate viral sepsis and inflammatory-induced lung injury which can lead to other complications, including pneumonitis, pneumonia, acute respiratory distress syndrome (ARDS), pneumonia, respiratory failure, septic shock, organ failure and death.

COVID-19 emerged recently in China and quickly has spread worldwide, resulting in >218,175 confirmed cases and 8,937 deaths as of Mar. 18, 2020. There is an urgent need for safe and effective therapeutic and prophylactic agents against betacoronavirus infections, such as infections by SARS-CoV-2, SARS-CoV-1, MERS-CoV, and related viruses.

### SUMMARY OF THE INVENTION

The invention features the use of nitro-aminoadamantane compounds for the treatment and prophylaxis of symptoms of beta-coronavirus infections, such as infections by SARS-CoV-2, SARS-CoV-1, MERS-CoV, and related viruses.

In a first aspect, the invention features a method of treating a betacoronavirus infection in a human subject, the method including administering to the subject a therapeutically effective amount of a nitro-aminoadamantane compound or a pharmaceutically acceptable salt thereof.

The invention further features a method of ameliorating one or more symptoms of a betacoronavirus infection in a human subject, the method including administering to the subject a therapeutically effective amount of a nitro-aminoadamantane compound or a pharmaceutically acceptable salt thereof. The one or more symptoms can include fever, cough, shortness of breath, bilateral lung involvement with ground-glass opacity (observable from computed tomography images), or any other symptom described herein. The one or more symptoms can be reduced either in their frequency by 10%, 20%, 30%, or 50% relative to control subjects of the same age and having the same comorbidities as untreated. In particular embodiments, the symptom of betacoronavirus infection is a post-infection reduction in mental clarity and/or inability to focus (i.e., “brain fog”).

The invention also features a method of inhibiting the progression of a betacoronavirus infection in a human subject, the method including administering to the subject a therapeutically effective amount of a nitro-aminoadamantane compound or a pharmaceutically acceptable salt thereof. For example, the risk of progression to pneumonitis, pneumonia, acute respiratory distress syndrome, respiratory failure, septic shock, organ failure, cytokine storm, and/or death can be inhibited by 10%, 20%, 30%, or 50% relative to control subjects of the same age and having the same comorbidities as untreated.

In a related aspect, the invention features a method of reducing the likelihood of betacoronavirus infection in a human subject at risk thereof, the method including administering to the subject a therapeutically effective amount of a nitro-aminoadamantane compound or a pharmaceutically acceptable salt thereof. The subject at risk can be a subject know to have been in contact with an infected person or in contact with a location previously occupied by an infected person. In certain embodiments, the subject at risk is under quarantine. The likelihood of betacoronavirus infection can be reduced by 10%, 20%, 30%, or 50% relative to control subjects of the same age and having the same comorbidities, as untreated control subjects.

In particular embodiments of any of the above methods, the risk of hospitalization of the subject is reduced. In other embodiments of any of the above methods, the duration of hospitalization is reduced.

In embodiments of any of the above methods, the administration occurs between once per week to three times per day. For example, the administration can be once per day or twice per day. The administration can occur over a treatment period, e.g., of about 1 day to about 21 days (e.g., 1 to 14 days, 7±3 days, 10±4 days, 15±6 days), or from about 1 week to about 6 weeks, or over a longer treatment period, if necessary.

In particular embodiments of the above methods, the subject is being hospitalized or quarantined for the betacoronavirus infection.

In some embodiments of any of the above methods, the subject has a pre-existing condition that places the subject at higher risk of pneumonitis, pneumonia, acute respiratory distress syndrome, pneumonia, respiratory failure, septic shock, organ failure, cytokine storm, or death. For example, the subject at higher risk can be one having a pre-existing condition selected from cardiovascular disease, diabetes, chronic respiratory disease, hypertension, and obesity.

In other embodiments of any of the above methods, the subject is at least 20, 30, 40, 50, 60, 70, or 80 years old.

In one embodiment of any of the above methods, the betacoronavirus is SARS-CoV-2.

In another embodiment of any of the above methods, the betacoronavirus is SARS-CoV-1.

In still another embodiment of any of the above methods, the betacoronavirus is MERS-CoV.

In still another embodiment of any of the above methods, the betacoronavirus is a mutated form of SARS-CoV-1 or SARS-CoV-2.

In one embodiment of any of the above methods, the nitro-aminoadamantane compound is a compound of any one of formulas (I)-(V) (described herein).

In one particular embodiment of any of the above methods, the nitro-aminoadamantane compound is selected from:

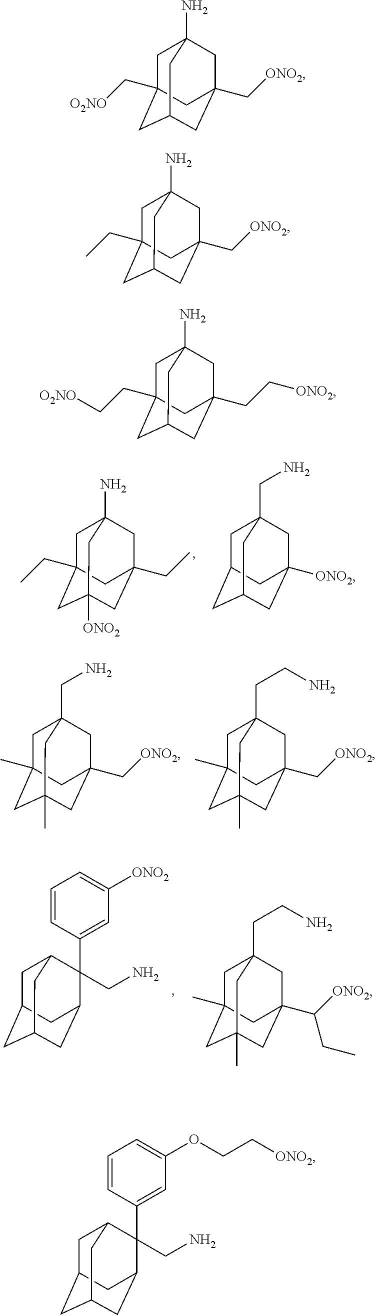

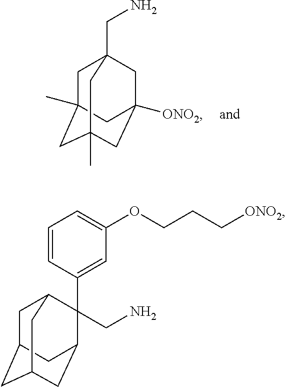

and pharmaceutically acceptable salts thereof.

### Definitions

As used herein, the term “about” means+/−10% of the recited value.

As used herein, by “administration” or“administering” is meant a method of giving a dosage of a nitro-aminoadamantane compound to a subject. The nitro-aminoadamantane compounds utilized in the methods described herein can be administered, for example, orally, or by another other route described herein.

As used herein. “reducing the risk of pneumonitis” in a subject refers to reducing the frequency of pneumonitis in subjects treated according to the methods of the invention. The reduction is in comparison to control subjects of the same age and condition (e.g., comorbidities) that are untreated.

The frequency of pneumonitis can be reduced by 10%, 20%, 30%, or 50% relative to the frequency of pneumonitis observed for the control subjects.

As used herein. “reducing the risk of acute respiratory distress syndrome” in a subject refers to reducing the frequency of acute respiratory distress syndrome in subjects treated according to the methods of the invention. The reduction is in comparison to control subjects of the same age and condition (e.g., comorbidities) that are untreated. The frequency of acute respiratory distress syndrome can be reduced by 10%, 20%, 30%, or 50% relative to the frequency of acute respiratory distress syndrome observed for the control subjects.

As used herein, “reducing the risk of respiratory failure” in a subject refers to reducing the frequency of respiratory failure in subjects treated according to the methods of the invention. The reduction is in comparison to control subjects of the same age and condition (e.g., comorbidities) that are untreated. The frequency of respiratory failure can be reduced by 10%, 20%, 30%, or 50% relative to the frequency of respiratory failure observed for the control subjects.

As used herein, “reducing the risk of pneumonia” in a subject refers to reducing the frequency or severity of pneumonia in subjects treated according to the methods of the invention. The reduction is in comparison to control subjects of the same age and condition (e.g., comorbidities) that are untreated. The frequency or severity of pneumonia can be reduced by 10%, 20%, 30%, or 50% relative to the frequency or severity of pneumonia observed for the control subjects.

As used herein, “reducing the risk of septic shock” in a subject refers to reducing the frequency of septic shock in subjects treated according to the methods of the invention. The reduction is in comparison to control subjects of the same age and condition (e.g., comorbidities) that are untreated. The frequency of septic shock can be reduced by 10%, 20%, 30%, or 50% relative to the frequency of septic shock observed for the control subjects.

As used herein, “reducing the risk of organ failure” in a subject refers to reducing the frequency of organ failure in subjects treated according to the methods of the invention. The reduction is in comparison to control subjects of the same age and condition (e.g., comorbidities) that are untreated. The frequency of organ failure can be reduced by 10%, 20%, 30%, or 50% relative to the frequency of organ failure observed for the control subjects.

As used herein, “reducing the risk of death” in a subject refers to reducing the frequency of death in subjects treated according to the methods of the invention. The reduction is in comparison to control subjects of the same age and condition (e.g., comorbidities) that are untreated. The frequency of death can be reduced by 10%, 20%, 30%, or 50% relative to the frequency of death observed for the control subjects.

As used herein, “reducing the risk of cytokine storm” in a subject refers to reducing the frequency of cytokine storm in subjects treated according to the methods of the invention. The reduction is in comparison to control subjects of the same age and condition (e.g., comorbidities) that are untreated. The frequency of cytokine storm can be reduced by 10%, 20%, 30%, or 50% relative to the frequency of cytokine storm observed for the control subjects.

As used herein, “reducing the risk of hospitalization” in a subject refers to reducing the frequency of hospitalization in subjects treated according to the methods of the invention. The reduction is in comparison to control subjects of the same age and condition (e.g., comorbidities) that are untreated. The frequency of hospitalization can be reduced by 10%, 20%, 30%, or 50% relative to the frequency of hospitalization observed for the control subjects.

As used herein, “reducing the duration of hospitalization” in a subject refers to reducing the duration of hospitalization in subjects treated according to the methods of the invention. The reduction is in comparison to control subjects of the same age and condition (e.g., comorbidities) that are untreated. The duration of hospitalization can be reduced by 10%, 20%, 30%, or 50% relative to the duration of hospitalization observed for the control subjects.

As used herein, a “therapeutically effective amount” refers to an amount of a nitro-aminoadamantane compound required to treat, ameliorate the symptoms of, inhibit the progression of, or reduce the likelihood of developing a betacoronavirus infection. The effective amount of a nitro-aminoadamantane compound used to practice the invention for therapeutic or prophylactic treatment of conditions caused by or contributed to by a betacoronavirus infection varies depending upon the manner of administration, the age, body weight, and general health of the subject. Ultimately, the attending physician will decide the appropriate amount and dosage regimen. Such amount is referred to as a “therapeutically effective amount.”

By “pharmaceutical composition” is meant any composition that contains a nitro-aminoadamantane compound combined with a pharmaceutically acceptable carrier that together is suitable for administration to a subject and that treats or prevents a betacoronavirus infection or reduces the severity of, or ameliorates, one or more symptoms associated with a betacoronavirus infection Pharmaceutical compositions useful in the methods of the invention can take the form of tablets, gelcaps, capsules, pills, powders, granulates, suspensions, and/or emulsions.

As used herein, the term “pharmaceutically acceptable carrier” refers to an excipient or diluent in a pharmaceutical composition. For example, a pharmaceutically acceptable carrier may be a vehicle capable of suspending or dissolving the active ingredients (e.g., a nitro-aminoadamantane compound). The pharmaceutically acceptable carrier can be compatible with the other ingredients of the formulation and not deleterious to the recipient. For oral administration, a solid carrier may be preferred.

As used herein, the term “treat” or “treating” includes administration of a nitro-aminoadamantane compound to a subject by any route, e.g., orally. The subject, e.g., a patient, can be one having a disorder (e.g., a disease or condition described herein), a symptom of a disorder, or a predisposition toward a disorder. Treatment is not limited to curing or complete healing, but can result in one or more of alleviating, relieving, altering, partially remedying, ameliorating, improving or affecting the betacoronavirus infection, reducing one or more symptoms of the betacoronavirus infection or the predisposition toward the betacoronavirus infection. In an embodiment the treatment (at least partially) alleviates or relieves symptoms related to a betacoronavirus infection. In one embodiment, the treatment reduces at least one symptom of the betacoronavirus infection or delays onset of at least one symptom of the betacoronavirus infection. The effect is beyond what is seen in the absence of treatment.

As used herein, the term “pharmaceutically acceptable salt” refers to salt forms (e.g., acid addition salts or metal salts) of the nitro-aminoadamantane compounds suitable for therapeutic use according to the methods of the invention.

As used herein, the term “nitro-aminoadamantane compound” refers to compounds including an adamantane moiety substituted by at least one amino group and at least one terminal nitrate group. The nitro-aminoadamantane compound used in the methods of the invention can be any nitro-aminoadamantane compound of formulas (I)-(V), or subgenera thereof.

The terms “halogen”, “halide” and “halo” refer to fluorine/fluoride, chlorine/chloride, bromine/bromide and iodine/iodide.

The term “alkyl” refers to a linear or branched, saturated monovalent hydrocarbon radical, wherein the alkyl group can optionally be substituted with one or more substituents as described herein. In certain embodiments, an alkyl group is a linear saturated monovalent hydrocarbon radical that has 1 to 10 (C₁₋₁₀) or 1 to 6 (C₁₋₆) carbon atoms, or is a branched saturated monovalent hydrocarbon radical that has 3 to 10 (C₃₋₁₀) or 3 to 6 (C₃₋₆) carbon atoms. As an example, the term “C₁₋₆ alkyl” refers to a linear saturated monovalent hydrocarbon radical of 1 to 6 carbon atoms or a branched saturated monovalent hydrocarbon radical of 3 to 6 carbon atoms. Linear C₁₋₆ and branched C₃₋₆ alkyl groups may also be referred to as “lower alkyl”. Non-limiting examples of alkyl groups include methyl, ethyl, propyl (including n-propyl and isopropyl), butyl (including all isomeric forms, such as n-butyl, isobutyl, seo-butyl and tert-butyl), pentyl (including all isomeric forms, such as n-pentyl), and hexyl (including all isomeric forms, such as n-hexyl).

The terms “alkylene” and “-alkyl-” refer to a divalent alkyl group, which can optionally be substituted with one or more substituents as described herein.

The term “heteroalkyl” refers to a linear or branched, saturated monovalent hydrocarbon group containing one or more heteroatoms independently selected from O, N and S. In some embodiments, one or more heteroatoms are in the main chain of the linear or branched hydrocarbon group. The terms “heteroalkylene” and “-heteroalkyl-” refer to a divalent heteroalkyl group. A heteroalkyl group and a -heteroalkyl-group can optionally be substituted with one or more substituents as described herein. Examples of heteroalkyl and -heteroalkyl- groups include without limitation —(CH₂)ₘ—(O or S)—(CH₂)ₙCH₃ and —(CH₂)ₘ(O or S)—(CH₂)ₚ—, wherein m is 1, 2 or 3, n is 0, 1 or 2, and p is 1, 2 or 3.

The term “alkoxy” refers to an —O-alkyl group, which can optionally be substituted with one or more substituents as described herein.

Examples of —O-heteroalkyl and —O-heteroalkyl- groups include without limitation ethylene glycol groups and polyethylene glycol (PEG) groups, including but not limited to —(OCH₂CH₂)ₘ—OR and —(OCH₂CH₂)ₘ—O—, wherein R is hydrogen or alkyl and n is 1, 2 or 3. It is understood that for a —O-heteroalkyl-ONO₂ group, when the —O-heteroalkyl- group is an ethylene glycol or PEG group, the terminal oxygen atom of the ethylene glycol or PEG group is part of the nitrate (—ONO₂) group. An —O-heteroalkyl group and an —O-heteroalkyl- group can optionally be substituted with one or more substituents as described herein.

The term “haloalkyl” refers to an alkyl group that is substituted with one or more halogen/halide atoms. A haloalkyl group can optionally be substituted with one or more additional substituents as described herein. Examples of haloalkyl groups include without limitation fluoroalkyl groups such as —CH₂F, —CHF₂ and —(CH₂)ₙCF₃, and perfluoroalkyl groups such as —CF₃ and —(CF₂)ₙCF₃, wherein n is 1, 2, 3, 4 or 5.

The term “-alkylaryl” refers to an alkyl group that is substituted with one or more aryl groups. An -alkylaryl group can optionally be substituted with one or more additional substituents as described herein.

The term “cycloalkyl” refers to a cyclic saturated, bridged or non-bridged monovalent hydrocarbon radical, which can optionally be substituted with one or more substituents as described herein. In certain embodiments, a cycloalkyl group has from 3 to 10 (C₃₋₁₀), or from 3 to 8 (C₃₋₈), or from 3 to 6 (C₃₋₆) carbon atoms. Non-limiting examples of cycloalkyl groups include cyclopropyl, cyclobutyl, cyclopentyl, cyclohexyl, cycloheptyl, cyclooctyl, norbornyl, decalinyl and adamantyl. The term “-cycloalkyl-” refers to a divalent cycloalkyl group, which can optionally be substituted with one or more substituents as described herein.

The terms “heterocyclyl” and “heterocyclic” refer to a monocyclic non-aromatic group or a multicyclic group that contains at least one non-aromatic ring, wherein at least one non-aromatic ring contains one or more heteroatoms independently selected from O, N and S. The non-aromatic ring containing one or more heteroatoms may be attached or fused to one or more saturated, partially unsaturated or aromatic rings. In certain embodiments, a heterocyclyl or heterocyclic group has from 3 to 10, or 3 to 8, or 3 to 6 ring atoms. In some embodiments, a heterocyclyl or heterocyclic group is a monocyclic, bicyclic or tricyclic ring system, which may include a fused or bridged ring system, and in which nitrogen or sulfur atoms can optionally be oxidized, nitrogen atoms can optionally be quaternized, and one or more rings may be fully or partially saturated, or aromatic. A heterocyclyl or heterocyclic group may be attached to the main structure at any heteroatom or carbon atom which results in the creation of a stable compound. Examples of heterocyclyl or heterocyclic groups include without limitation azepinyl, azetidinyl, aziridinyl, benzodioxanyl, benzodioxolyl, benzofuranonyl, benzopyranonyl, benzopyranyl, benzotetrahydrofuranyl, benzotetrahydrothienyl, benzothiopyranyl, O-carbolinyl, chromanyl, decahydroisoquinolinyl, dihydrobenzisothiazinyl, dihydrobenzisoxazinyl, dihydrofuryl, dihydropyranyl, dihydropyrazinyl, dihydropyridinyl, dihydropyrazolyl, dihydropyrimidinyl, dihydropyrrolyl, dioxolanyl, dithianyl, furanonyl, imidazolidinyl, imidazolinyl, indolinyl, indolizinyl, isobenzotetrahydrofuranyl, isobenzotetrahydrothienyl, isochromanyl, isoindolinyl, isothiazolidinyl, isoxazolidinyl, morpholinyl, octahydroindolyl, octahydroisoindolyl, oxazolidinonyl, oxazolidinyl, oxiranyl, piperazinyl, piperidinyl, 4-piperidonyl, pyrrolidinyl, pyrrolinyl, quinuclidinyl, tetrahydrofuryl, tetrahydrofuranyl (oxolanyl), tetrahydroisoquinolinyl, tetrahydropyranyl, tetrahydrothienyl (tetrahydrothiophenyl, thiolanyl), thiamorpholinyl (thiomorpholinyl), thiazolidinyl and 1,3,5-trithianyl. The term “-heterocyclyl-” refers to a divalent heterocyclyl group. A heterocyclyl or heterocyclic group, and a -heterocyclyl- group, can optionally be substituted with one or more substituents as described herein.

The term “aryl” refers to a monocyclic aromatic hydrocarbon group or a multicyclic group that contains at least one aromatic hydrocarbon ring. In certain embodiments, an aryl group has from 6 to 10 ring atoms. Non-limiting examples of aryl groups include phenyl, naphthyl, fluorenyl, azulenyl, anthryl, phenanthryl, biphenyl and terphenyl. The aromatic hydrocarbon ring of an aryl group may be attached or fused to one or more saturated, partially unsaturated or aromatic rings—e.g., dihydronaphthyl, indenyl, indanyl and tetrahydronaphthyl (tetralinyl). The term “-aryl-” refers to a divalent aryl group. An aryl group and an -aryl- group can optionally be substituted with one or more substituents as described herein.

The term “heteroaryl” refers to a monocyclic aromatic group or a multicyclic group that contains at least one aromatic ring, wherein at least one aromatic ring contains one or more heteroatoms independently selected from O, N and S. The heteroaromatic ring may be attached or fused to one or more saturated, partially unsaturated or aromatic rings that may contain only carbon atoms or that may contain one or more heteroatoms. A heteroaryl group may be attached to the main structure at any heteroatom or carbon atom which results in the creation of a stable compound. In certain embodiments, a heteroaryl group has from 5 to 10 ring atoms. Examples of monocyclic heteroaryl groups include without limitation pyrrolyl, pyrazolyl, pyrazolinyl, imidazolyl, oxazolyl, isoxazolyl, thiazolyl, thiadiazolyl, isothiazolyl, furanyl, thienyl (thiophenyl), oxadiazolyl, triazolyl, tetrazolyl, pyridyl, pyridonyl, pyrazinyl, pyrimidinyl, pyridazinyl, pyridazinonyl and triazinyl. Non-limiting examples of bicyclic heteroaryl groups include indolyl, benzothiazolyl, benzothiadiazolyl, benzoxazolyl, benzisoxazolyl, benzothienyl (benzothiophenyl), quinolinyl, tetrahydroisoquinolinyl, isoquinolinyl, benzimidazolyl, benzotriazolyl, indolizinyl, benzofuranyl, isobenzofuranyl, chromonyl, coumarinyl, cinnolinyl, quinazolinyl, quinoxalinyl, indazolyl, naphthyridinyl, phthalazinyl, quinazolinyl, purinyl, pyrrolopyridinyl, furopyridinyl, thienopyridinyl, dihydroisoindolyl and tetrahydroquinolinyl. Examples of tricyclic heteroaryl groups include without limitation carbazolyl, benzindolyl, dibenzofuranyl, phenanthrollinyl, acridinyl, phenanthridinyl, xanthenyl and phenothiazinyl. The term “-heteroaryl-” refers to a divalent heteroaryl group. A heteroaryl group and a -heteroaryl- group can optionally be substituted with one or more substituents as described herein.

Each group described herein (including without limitation monovalent and divalent alkyl, heteroalkyl, —O-alkyl, —O-heteroalkyl, alkylaryl, cycloalkyl, heterocyclyl, aryl and heteroaryl), whether as a primary group or as a substituent group, can optionally be substituted with one or more substituents. In certain embodiments, each group described herein can optionally be substituted with 1, 2, 3, 4, 5 or 6 substituents independently selected from halide, cyano, nitro, nitrate, hydroxyl, sulfhydryl (—SH), —NH₂, —OR¹¹, —SR¹¹, —NR¹²R¹³, —C(═O)R¹¹, —C(═O)OR¹¹, —OC(═O)R¹¹, —C(═O)NR¹²R¹³, —NR¹²C(═O)R¹¹, —OC(═O)OR¹¹, —OC(═O)NR¹²R¹³, —NR¹²C(═O)OR¹¹, —NR¹¹C(═O)NR¹²R¹³, alkyl, haloalkyl, alkoxy, cycloalkyl, heterocyclyl, aryl and heteroaryl, wherein:

R¹¹ in each occurrence independently is hydrogen, alkyl, cycloalkyl, heterocyclyl, aryl or heteroaryl; and

R¹² and R¹³ in each occurrence independently are hydrogen, alkyl, cycloalkyl, heterocyclyl, aryl or heteroaryl, or R¹² and R¹³ and the nitrogen atom to which they are connected form a heterocyclic or heteroaryl ring.

Other features and advantages of the invention will be apparent from the Drawings, the Detailed Description, and the claims.

### BRIEF DESCRIPTION OF THE DRAWINGS

FIG. 1 depicts the structures of the compounds tested in Example 5.

FIGS. 2A-2C show that NMT5 S-nitrosylates ACE2 and inhibits SARS-CoV-2 pseudoviral entry into HeLa-ACE2 cells. FIG. 2A is a picture showing HeLa-ACE2 cells were treated with 10 μM NMT3 or 5 μM NMT5. After 1 h, cell lysates were subjected to biotin-switch assay for protein S-nitrosylation, detected by immunoblotting with anti-ACE2 antibody. FIG. 2B is a graph depicting the ratio of SNO-ACE2/input ACE2 protein. Data are mean±s.e.m., **P<0.01, ns: not significant, n=4 biological replicates. FIG. 2C is a graph showing NMT5 inhibited SARS-CoV-2 pseudoviral entry in a dose-dependent manner. HeLa-ACE2 cells were inoculated with SARS-CoV-2 or VSV-G (control) pseudovirus particles in the presence and absence of NMT3 or NMT5. After 48 h, viral transduction efficiency was monitored by luciferase activity. Data are mean±s.e.m., *P<0.05, **P<0.01, ****P<0.0001, n=3 or 4 biological replicates.

FIGS. 3A and 3B depict a model of protein S-nitrosylation of ACE2 and NMT5 mechanism of action in inhibiting infection and propagation of SARS-CoV-2. FIG. 3A shows a schematic of SNO-ACE2-mediated inhibition of SARS-CoV-2 entry. Left: Pathogenic SARS-CoV-2 uses its spike protein to bind to the host cell surface receptor ACE2, facilitating viral entry into host cells. Right: S-Nitrosylation of ACE2 (forming SNO-ACE2) interferes with binding to spike protein, thus abrogating SARS-CoV-2 entry into host cells. FIG. 3B depicts a schematic of the dual action of nitro-aminoadamantane compound NMT5, which mediates S-nitrosylation of ACE2 to inhibit viral entry, and blockade of the viral E protein ion channel to inhibit propagation of SARS-CoV-2.

### DETAILED DESCRIPTION OF THE INVENTION

The invention features methods for treating or preventing infections by betacoronavirus pathogens, including SARS-CoV-1 (that caused epidemics in China in 2002-2003), MERS-CoV (that affected Saudi Arabia and neighbor countries in 2012-2013), and SARS-CoV-2 (which emerged recently in China and quickly has spread worldwide). The methods include administering a nitro-aminoadamantane compound to a subject suffering, or at risk of, the infection.

With similar clinical presentations as SARS and MERS, the most common symptoms of COVID-19 are fever, fatigue, and respiratory symptoms, including cough, sore throat and shortness of breath. Such infections are characterized by high-levels of proinflammatory cytokines resulting in a “cytokine storm” that likely plays a major role in the pathogenesis of these infections. This so-called “cytokine storm” can initiate viral sepsis and inflammatory-induced lung injury which lead to other complications including pneumonitis, pneumonia, acute respiratory distress syndrome (ARDS), respiratory failure, septic shock, organ failure and death.

Compounds in the aminoadamantane family are generally known to block proton-carrying ion channels in envelope viruses, such as SARS-CoV-2, causing COVID-19 respiratory diseases. Moreover, nitric oxide (NO) and related compounds have been reported to affect inhibit these viruses. We reasoned in a novel fashion, and not obvious to one skilled in the art, that the targeted delivery of NO-related species to the virus would avoid side effects of NO-Ike drugs. For this purpose, we devised a series of nitro-aminoadamantane compounds, with the aminoadamantane moiety that can act as a “guided missile” to enter the viral envelope channel and then deliver a “warhead” of a nitro group directly to the virus to disrupt the virus. This approach increases efficacy while reducing the risk of clinical issues of off-target effects of the nitro group.

In “COVID-long haulers,” symptoms of betacoronavirus infection persist post-infection. Symptoms can include a reduction in mental clarity and/or inability to focus (i.e., “brain fog”). Nitro-aminoadamantane compounds can be used to both improve brain function post-infection and to protect from subjects from brain injury caused by the virus. In this regard, excessive levels of glutamate are known to be released or not taken up by astrocytes in the face of severe viral infections, including coronaviruses, leading to glutamate-related toxicity or excitotoxicity (Brison et al., J Virol. 85(23):12484-73 (2011); Soung et al., Trends Mol Med. 24(11):950-962 (2018)). Astrocytes release (or do not take up) glutamate as they normally do to clear the synaptic cleft after synaptic transmission or after injury to the CNS (Talantova et al., Proc Natl Acad Sci USA 110:E2518-2527 (2013); Akhtar et al., Nature Comm 7, 10242 (2015)). Because nitro-aminoadamantane compounds can act on glutamate receptors to ameliorate or prevent glutamate-mediated damage to neurons that betacoronavirus infection could cause in the brain.

Provided herein are methods of using nitro-aminoadamantane compounds for treating, ameliorating the symptoms of, inhibiting progression of, or reducing the likelihood of developing a betacoronavirus infection in a subject.

The nitro-aminoadamantane compound used in the methods of the invention can be a compound of any of formulas (I)-(V):

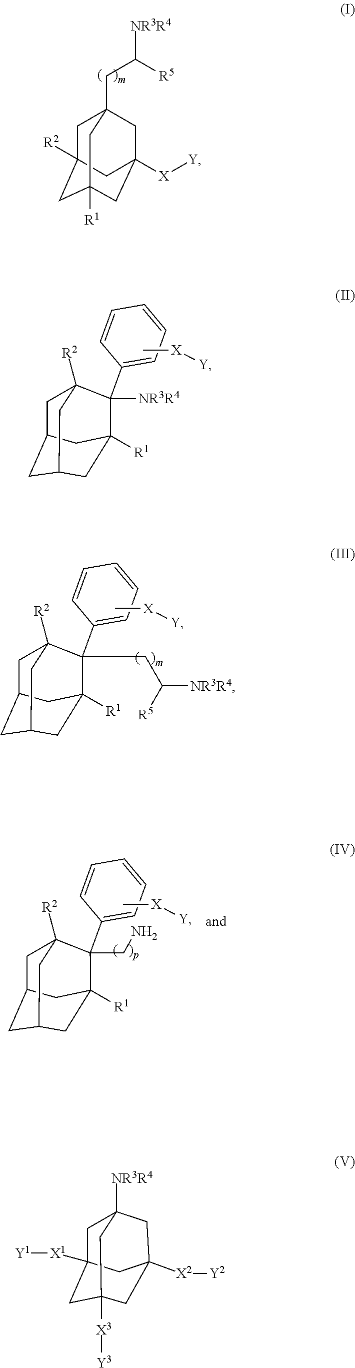

wherein in formulas (I)-(IV), Y is a nitrate-containing group and R1, R2, R3, R4, R5, X, p, and m are as defined elsewhere herein. In formula (V) each of Y¹, Y², and Y³, is optionally a nitrate-containing group and Y¹, Y², Y³, X¹, X², X³, R³, and R⁴ m are as defined elsewhere herein

### Niro-Aminoadamantane Compounds

The nitro-aminoadamantane compounds used in the methods of the invention can be synthesized using methods analogous to those described in U.S. Pat. No. 7,326,730 and PCT Publication No. WO2019104020, each of which is incorporated herein in its entirety.

Compounds of Formula (I)-(IV)

The nitro-aminoadamantane compound used in the methods of the invention can be a compound of formula (I):

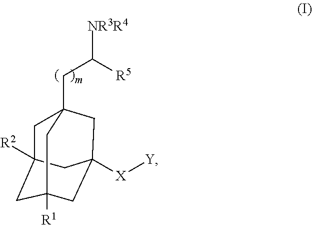

or a pharmaceutically acceptable sat thereof, wherein:

R¹ and R² independently are hydrogen, halide, linear or branched alkyl, linear or branched heteroalkyl, linear or branched alkoxy, linear or branched —O-heteroalkyl, cycloalkyl, heterocyclyl, aryl or heteroaryl, each of which can optionally be substituted;

R³ and R⁴ independently are hydrogen or linear or branched C₁-C₆ alkyl, or R³, R⁴ and the nitrogen atom to which they are attached form a 3-8-membered heterocyclic ring;

R⁵ is hydrogen or linear or branched C₁-C₆ alkyl;

X is bond, linear or branched -alkyl-, linear or branched -heteroalkyl-, linear or branched —O-alkyl-, linear or branched —O-heteroalkyl-, —(CH₂)ⱼ-cycloalkyl-(CH₂)ₖ—, —(CH₂)ⱼ-heterocyclyl-(CH₂)ₖ—, —(CH₂)ⱼ-aryl-(O)ₙ—(CH₂)ₖ— or —(CH₂)ⱼ-heteroaryl-(O)ₕ—(CH₂)ₖ—, each of which can optionally be substituted;

Y is —ONO₂ or

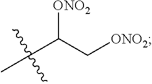

m is 0, 1, 2, 3, 4 or 5; j is 0, 1, 2or 3; k is 0, 1, 2or 3; and h is 0or 1.

In certain embodiments, the nitro-aminoadamantane compound used in the methods of the invention is further described by formula (Ia):

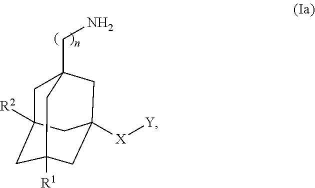

or a pharmaceutically acceptable salt thereof, wherein R¹, R², X and Y are as defined for formula (I); and n is 1, 2, 3, 4, 5 or 6.

In certain embodiments, the nitro-aminoadamantane compound used in the methods of the invention is further described by formula (IA):

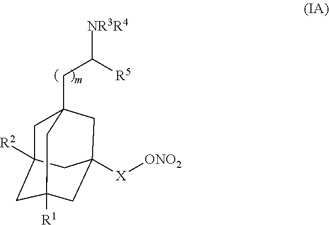

or a pharmaceutically acceptable salt thereof, wherein R¹, R², R³, R⁴, R⁵, X and m are as defined for formula (I).

In certain embodiments, the nitro-aminoadamantane compound used in the methods of the invention is further described by formula (IA-a):

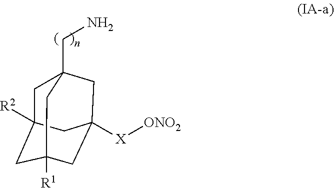

or a pharmaceutically acceptable salt thereof, wherein R¹, R² and X are as defined for formula (I); and n is 1, 2, 3, 4, 5 or 6.

In certain embodiments, the nitro-aminoadamantane compound used in the methods of the invention is further described by formula (IB):

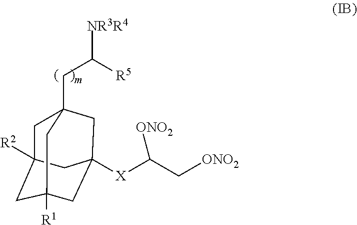

or a pharmaceutically acceptable salt thereof, wherein R¹, R², R³, R⁴, R⁵, X and m are as defined for formula (I).

In certain embodiments, the nitro-aminoadamantane compound used in the methods of the invention is further described by formula (IB-a):

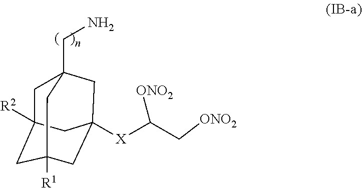

or a pharmaceutically acceptable salt thereof, wherein R¹, R² and X are as defined for formula (I); and n is 1, 2, 3, 4, 5 or 6.

In some embodiments, X of the compounds of formula (I) and subgenera thereof is bond, linear or branched C₁-C₆ or C₁-C₃-alkyl-, or linear or branched C₁-C₆ or C₁-C₃—O-alkyl-. In certain embodiments, X of the compounds of Formula I and subgenuses thereof is bond or linear or branched C₁-C₃-alkyl- [e.g., —CH₂—, —(CH₂)₂—, —ĆHCH₃, —(CH₂)₃—, —ĆHCH₂CH₃, —CH₂ĆHCH₃ or —CH(CH₃)CH₂—].

The nitro-aminoadamantane compound used in the methods of the invention can be a compound of formula (II) or formula (III):

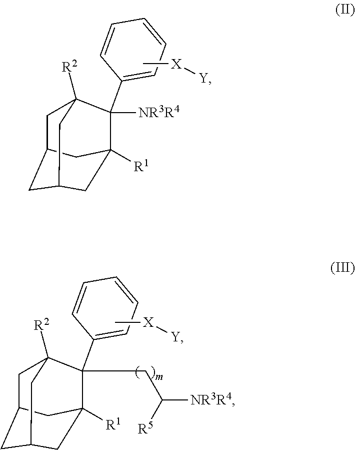

or a pharmaceutically acceptable salt thereof, wherein:

R¹ and R² independently are hydrogen, halide, linear or branched alkyl, linear or branched heteroalkyl, linear or branched alkoxy, linear or branched —O-heteroalkyl, cycloalkyl, heterocyclyl, aryl or heteroaryl, each of which can optionally be substituted;

R³ and R⁴ independently are hydrogen or linear or branched C₁-C₆ alkyl, or R³, R⁴ and the nitrogen atom to which they are attached form a 3-8-membered heterocyclic ring;

R⁵ is hydrogen or linear or branched C₁-C₆ alkyl;

X is bond, linear or branched -alkyl-, linear or branched -heteroalkyl-, linear or branched —O-alkyl-, linear or branched —O-heteroalkyl-, —(CH₂)ⱼ-cycloalkyl-(CH₂)ₖ—, —(CH₂)ⱼ-heterocyclyl-(CH₂)ₖ—, —(CH₂)ⱼ-aryl-(O)ₙ—(CH₂)ₖ— or —(CH₂)ⱼ-heteroaryl-(O)ₕ—(CH₂)ₖ—, each of which can optionally be substituted;

Y is —ONO₂ or

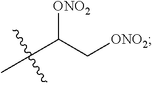

m is 0, 1, 2, 3, 4 or 5; j is 0, 1, 2 or 3; k is 0, 1, 2 or 3; and h is 0 or 1.

The nitro-aminoadamantane compound used in the methods of the invention can be a compound of formula (IV):

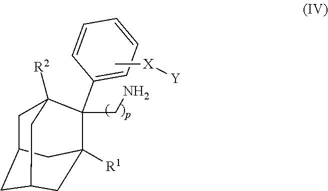

or a pharmaceutically acceptable salt thereof, wherein R¹, R², X and Y are as defined for formulas (II) and (III); and p is 0, 1, 2, 3, 4, 5 or 6.

In certain embodiments, the nitro-aminoadamantane compound used in the methods of the invention is further described by formula (IV-a):

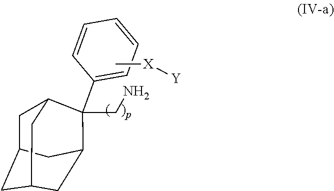

or a pharmaceutically acceptable salt thereof, wherein X and Y are as defined for formulas (II) and (III); and p is 0, 1, 2, 3, 4, 5 or 6.

In certain embodiments, the nitro-aminoadamantane compound used in the methods of the invention is further described by formula (II-a) or formula (Ill-a):

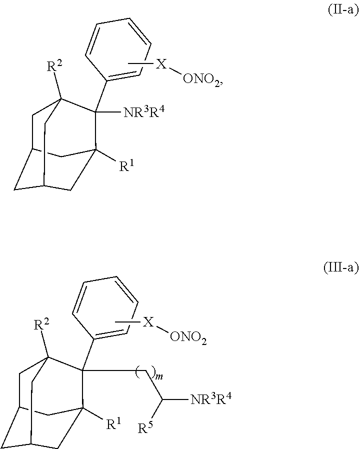

or a pharmaceutically acceptable salt thereof, wherein R¹, R², R³, R⁴, R⁵, X and m are as defined for formulas (II) and (III).

In certain embodiments, the nitro-aminoadamantane compound used in the methods of the invention is further described by formula (IVA):

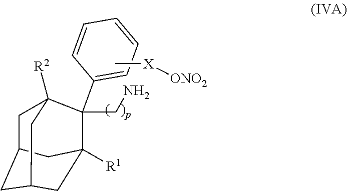

or a pharmaceutically acceptable salt thereof, wherein R¹, R² and X are as defined for formulas (II) and (III); and p is 0, 1, 2, 3, 4, 5 or 6.

In certain embodiments, the nitro-aminoadamantane compound used in the methods of the invention is further described by formula (IVA-a):

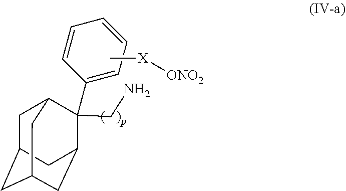

or a pharmaceutically acceptable salt thereof, wherein X is as defined for formulas (II) and (III); and p is 0, 1, 2, 3, 4, 5 or 6.

In certain embodiments, the nitro-aminoadamantane compound used in the methods of the invention is further described by formula (IIB) or formula (IIIB):

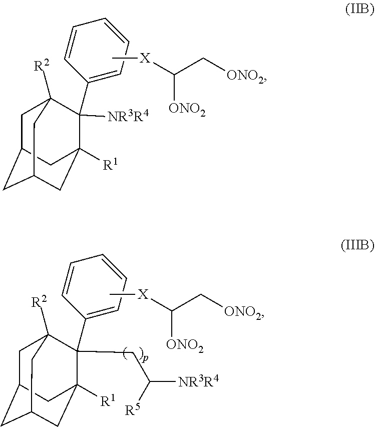

or a pharmaceutically acceptable salt thereof, wherein R¹, R², R³, R⁴, R⁵, X and m are as defined for formulas (II) and (III).

In certain embodiments, the nitro-aminoadamantane compound used in the methods of the invention is further described by formula (IVB):

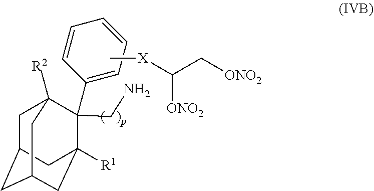

or a pharmaceutically acceptable salt thereof, wherein R¹, R² and X are as defined for formulas (II) and (III); and p is 0, 1, 2, 3, 4, 5 or 6.

In certain embodiments, the nitro-aminoadamantane compound used in the methods of the invention is further described by formula (IVB-a):

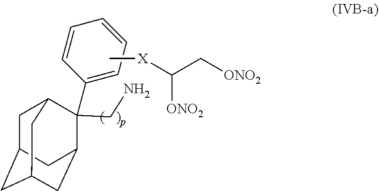

or a pharmaceutically acceptable salt thereof, wherein X is as defined for formulas (II) and (III); and p is 0, 1, 2, 3, 4, 5 or 6.

For the compounds of formulas (II) and (III), and subgenera thereof, the compounds of Formula III and subgenuses thereof, and the compounds of Formula IV and subgenuses thereof, the —X—Y, —X—ONO₂ or —X—CH(ONO₂)CH₂—ONO₂ moiety can be attached to an ortho position, a meta position or the para position of the phenyl ring. In certain embodiments, the —X—Y, —X—ONO₂ or —X—CH(ONO₂)CH₂—ONO₂ moiety is attached to a meta position of the phenyl ring.

For the compounds of formulas (II), (III), (IV), and subgenera thereof, in some embodiments X is bond, linear or branched C₁-C₆ or C₁-C₃-alkyl-, or linear or branched C₁-C₆ or C₁-C₃—O-alkyl-. In certain embodiments, X is bond or linear or branched C₁-C₃—O-alkyl- [e.g., —O—CH₂—, —O—(CH₂)₂—, —O—OHCH₃, —O—(CH₂)₃—, —O—OHCH₂CH₃, —O-CH₂ĆHCH₃ or —O—CH(CH₃)CH₂—].

For the compounds of formulas (I), (II) and (III), and subgenera thereof, examples of 3-8-membered, nitrogen-containing heterocyclic rings include without limitation aziridinyl, azetidinyl, pyrrolidinyl, piperidinyl, morpholinyl, piperazinyl, azepanyl and azocanyl, where the second ring nitrogen atom of a piperazinyl group can optionally be substituted with a linear C₁-C₃ alkyl group or a —(C═O)R group, wherein R is hydrogen or linear C₁-C₃ alkyl. In certain embodiments, R³, R⁴ and the nitrogen atom to which they are attached form a 3-8-membered heterocyclic ring.

For the compounds of formulas (I), (III) and (IV), and subgenera thereof, in certain embodiments m is 0, 1 or 2; n is 1, 2 or 3; and p is 0, 1, 2 or 3.

For the compounds of formulas (I), (II) and (III), and subgenera thereof, in some embodiments both R³ and R⁴ are hydrogen. In other embodiments, one of R³ and R⁴ is hydrogen, and the other is linear or branched C₁-C₃ alkyl. In certain embodiments, one of R³ and R⁴ is hydrogen, and the other is methyl or ethyl. In yet other embodiments, R³ and R⁴ independently are linear C₁-C₃ alkyl (e.g., methyl or ethyl), optionally the same alkyl group.

For the compounds of formulas (I) and (III), and subgenera thereof, in some embodiments R⁵ is hydrogen. In other embodiments, R⁵ is linear or branched C₁-C₃ alkyl. In certain embodiments, R⁵ is methyl or ethyl.

For the compounds of formulas (I), (II), (III), and (IV), and subgenera thereof, in some embodiments R¹ and R² independently are hydrogen or linear or branched C₁-C₆ or C₁-C₃ alkyl. In certain embodiments, both R¹ and R² are hydrogen. In other embodiments, R¹ is hydrogen and R² is linear or branched C₁-C₆ or C₁-C₃ alkyl, or R² is hydrogen and R¹ is linear or branched C₁-C₆ or C₁-C₃ alkyl. In certain embodiments, R¹ is hydrogen and R² is methyl, ethyl or n-propyl, or R² is hydrogen and R¹ is methyl, ethyl or n-propyl. In yet other embodiments, R¹ and R² independently are linear or branched C₁-C₆ or C₁-C₃ alkyl, optionally the same alkyl group. In certain embodiments, R¹ and R² independently are methyl, ethyl or n-propyl, optionally the same alkyl group. In some embodiments, R¹ is hydrogen and R² is methyl or ethyl, or R² is hydrogen and R¹ is methyl or ethyl. In other embodiments, both R¹ and R² are methyl or ethyl.

For the compounds of formulas (I), (II), (III), and (IV), and subgenera thereof, in some embodiments the R¹ group, the R² group or the X group, or any combination or all thereof, independently are substituted with 1, 2 or 3 substituents selected from linear or branched C₁-C₆ or C₁-C₃ alkyl, haloalkyl, —OR⁶, —NR⁷R⁸, —ONO₂, —CN, —C(═O)R⁶, —C(═O)OR⁶, —OC(═O)R⁶, —C(═O)NR⁷R⁸, —NR⁷C(═O)R⁶, —OC(═O)OR⁶, —OC(═O)NR⁷R⁸, —NR⁷C(═O)OR⁸, —NR⁶C(═O)NR⁷R⁸, aryl and heteroaryl, or/and are substituted with 1 to 6 halogen (e.g., fluorine) or have all available hydrogen atoms replaced with halogen (e.g., fluorine), wherein R⁶ in each occurrence independently is hydrogen or linear or branched C₁-C₆ or C₁-C₃ alkyl; and R⁷ and R⁸ in each occurrence independently are hydrogen or linear or branched C₁-C₆ or C₁-C₃ alkyl, or R⁷, R⁸ and the nitrogen atom to which they are attached form a 3-8-membered ring. In certain embodiments, the R¹ group, the R² group or the X group, or any combination or all thereof, independently are monovalent or divalent fluoroalkyl or alkyl-ONO₂.

For the compounds of formulas (I), (II), (III), and (IV), and subgenera thereof, non-limiting examples of linear or branched C₁-C₆ alkyl groups include methyl, ethyl, n-propyl, isopropyl, n-butyl, isobutyl, sec-butyl, tert-butyl, n-pentyl and n-hexyl. Examples of linear or branched C₁-C₃ alkyl groups include methyl, ethyl, n-propyl and isopropyl.

For the compounds of formulas (I), (II), (III), and (IV), and subgenera thereof, in some embodiments X has 0, 1, 2, 3, 4, 5 or 6 carbon atoms. In certain embodiments, X has 0, 1, 2 or 3 carbon atoms.

Table 1 depicts representative compounds of formula (IA-a-i) to (IA-a-xx):

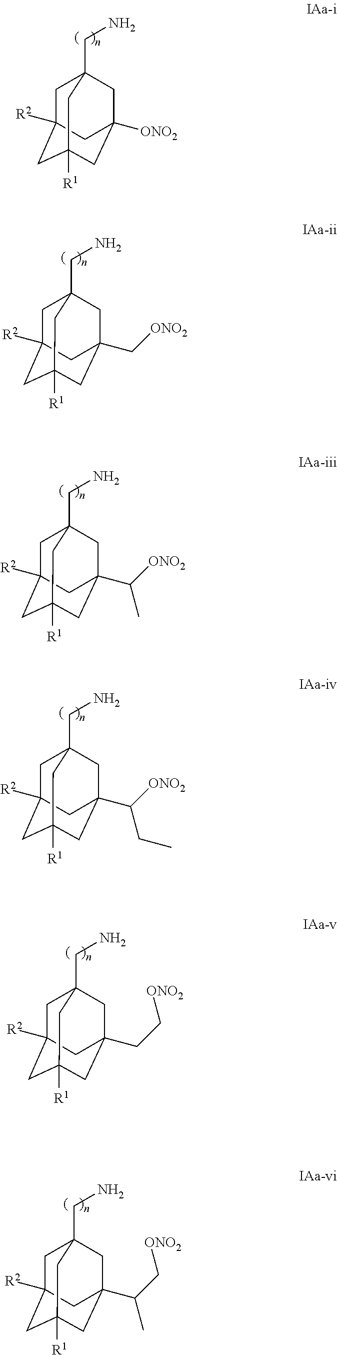

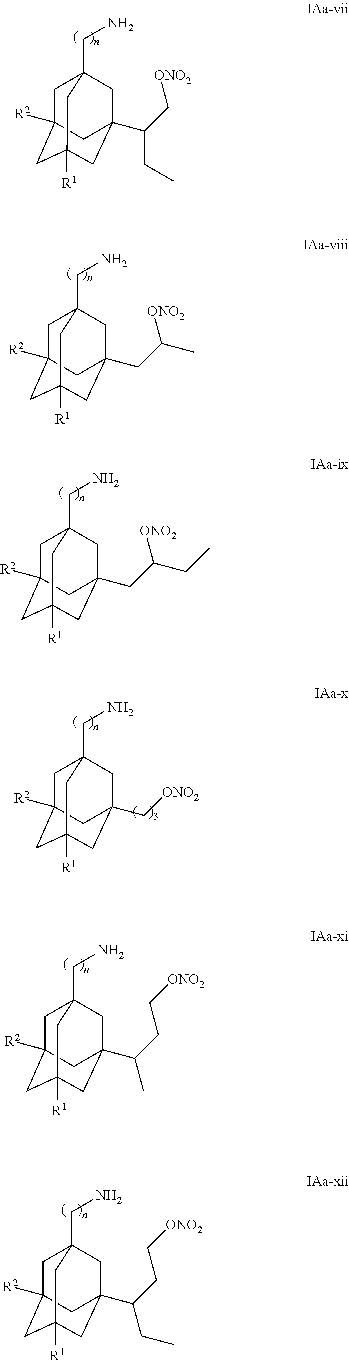

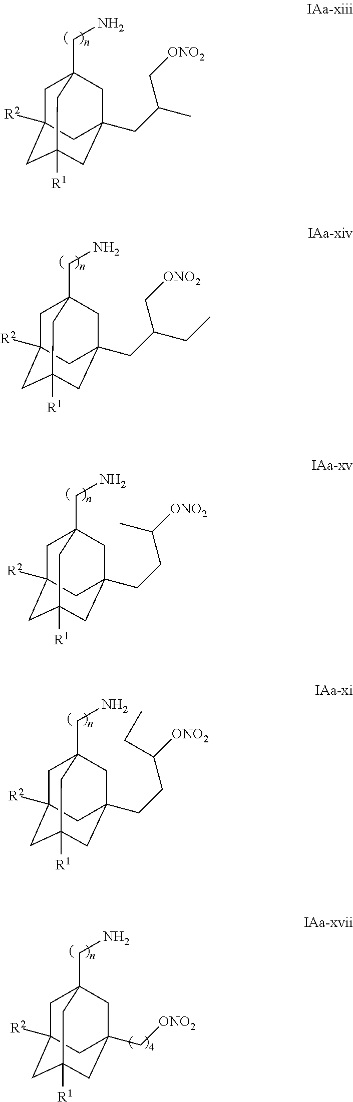

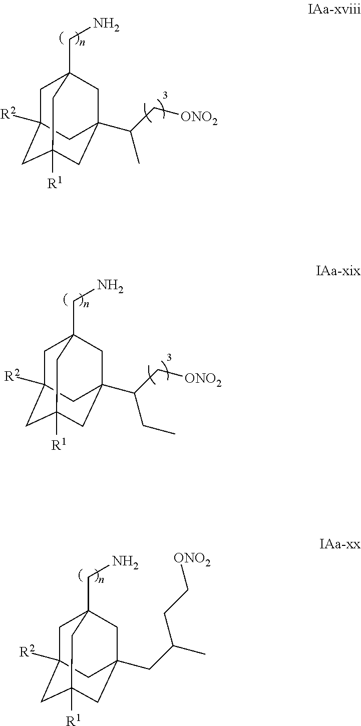

TABLE 1
For each subgenus of formula (IA-a-i) to (IA-a-xx):

| n | R¹ | R² |
|---|---|---|
| 1, 2 and 3 | methyl | methyl |
| 1, 2 and 3 | hydrogen | methyl |
| 1, 2 and 3 | methyl | hydrogen |
| 1, 2 and 3 | ethyl | ethyl |
| 1, 2 and 3 | hydrogen | ethyl |
| 1, 2 and 3 | ethyl | hydrogen |
| 1, 2 and 3 | n-propyl | n-propyl |
| 1, 2 and 3 | hydrogen | n-propyl |
| 1, 2 and 3 | n-propyl | hydrogen |
| 1, 2 and 3 | isopropyl | isopropyl |
| 1, 2 and 3 | hydrogen | isopropyl |
| 1, 2 and 3 | isopropyl | hydrogen |
| 1, 2 and 3 | n-butyl | n-butyl |
| 1, 2 and 3 | hydrogen | n-butyl |
| 1, 2 and 3 | n-butyl | hydrogen |
| 1, 2 and 3 | isobutyl | isobutyl |
| 1, 2 and 3 | hydrogen | isobutyl |
| 1, 2 and 3 | isobutyl | hydrogen |
| 1, 2 and 3 | sec-butyl | sec-butyl |
| 1, 2 and 3 | hydrogen | sec-butyl |
| 1, 2 and 3 | sec-butyl | hydrogen |
| 1, 2 and 3 | —CH₂—ONO₂ | —CH₂—ONO₂ |
| 1, 2 and 3 | hydrogen | —CH₂—ONO₂ |
| 1, 2 and 3 | —CH₂—ONO₂ | hydrogen |
| 1, 2 and 3 | —(CH₂)₂—ONO₂ | —(CH₂)₂—ONO₂ |
| 1, 2 and 3 | hydrogen | —(CH₂)₂—ONO₂ |
| 1, 2 and 3 | —(CH₂)₂—ONO₂ | hydrogen |
| 1, 2 and 3 | —(CH₂)₃—ONO₂ | —(CH₂)₃—ONO₂ |
| 1, 2 and 3 | hydrogen | —(CH₂)₃—ONO₂ |
| 1, 2 and 3 | —(CH₂)₃—ONO₂ | hydrogen |
| 1, 2 and 3 | —CH₂CH(ONO₂)CH₃ | —CH₂CH(ONO₂)CH₃ |
| 1, 2 and 3 | hydrogen | —CH₂CH(ONO₂)CH₃ |
| 1, 2 and 3 | —CH₂CH(ONO₂)CH₃ | hydrogen |
| 1, 2 and 3 | —CH₂CH(CH₃)CH₂—ONO₂ | —CH₂CH(CH₃)CH₂—ONO₂ |
| 1, 2 and 3 | hydrogen | —CH₂CH(CH₃)CH₂—ONO₂ |
| 1, 2 and 3 | —CH₂CH(CH₃)CH₂—ONO₂ | hydrogen |

In certain embodiments, the nitro-aminoadamantane compound used in the methods of the invention is further described by a subgenus of formula (IA-a-i) to (IA-a-xx), or a pharmaceutically acceptable salt thereof.

In certain embodiments, the nitro-aminoadamantane compound used in the methods of the invention is selected from:

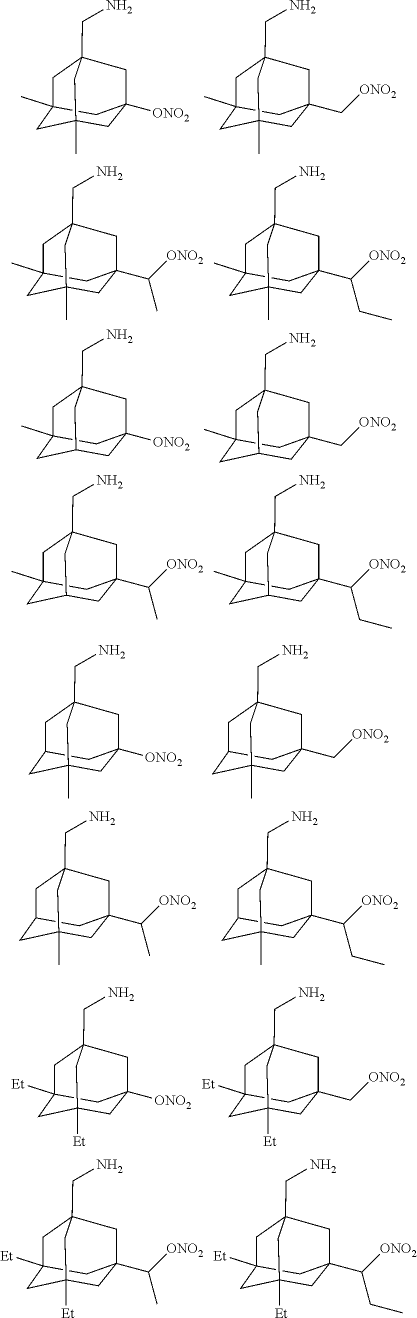

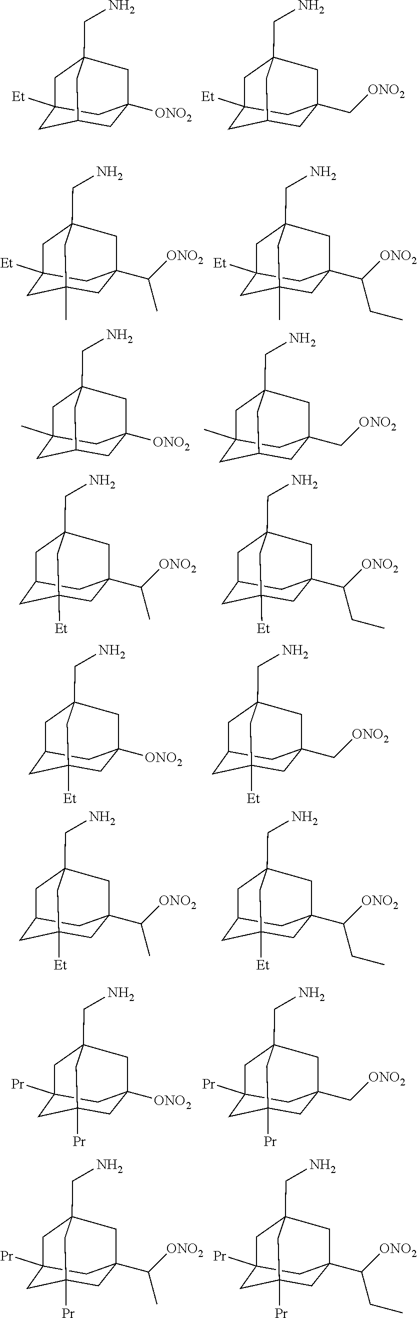

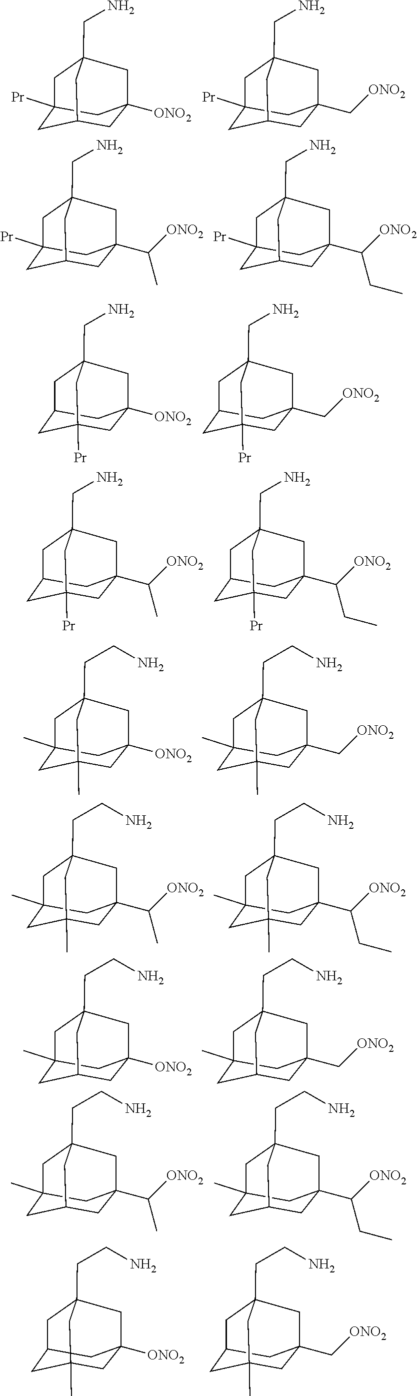

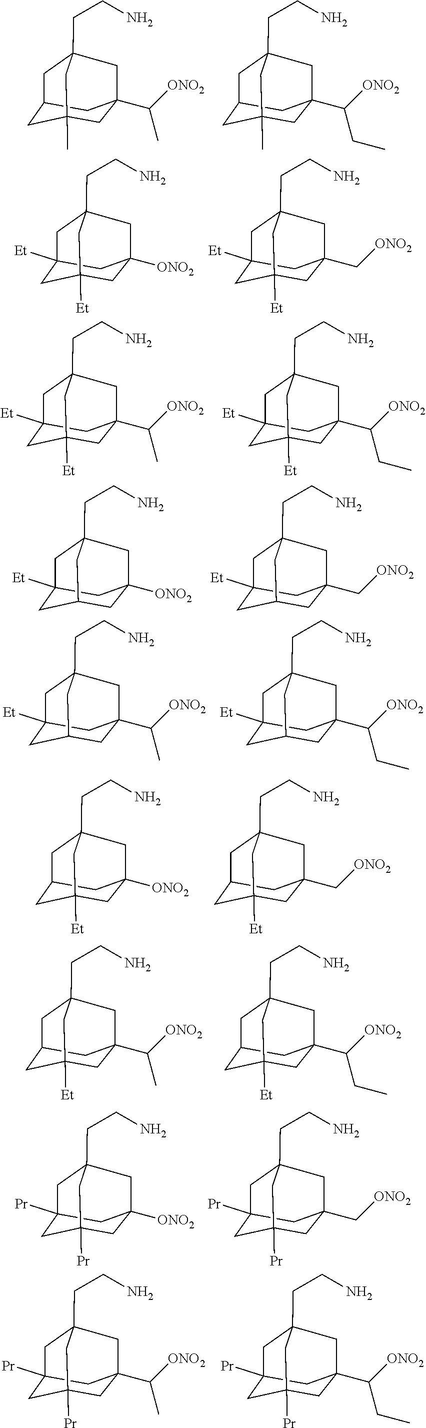

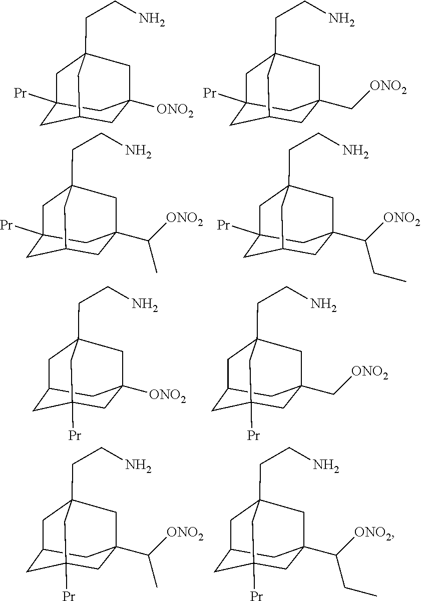

and pharmaceutically acceptable salts thereof.

Table 2 depicts representative compounds of formula (IB-a-i) to (IB-a-vii):

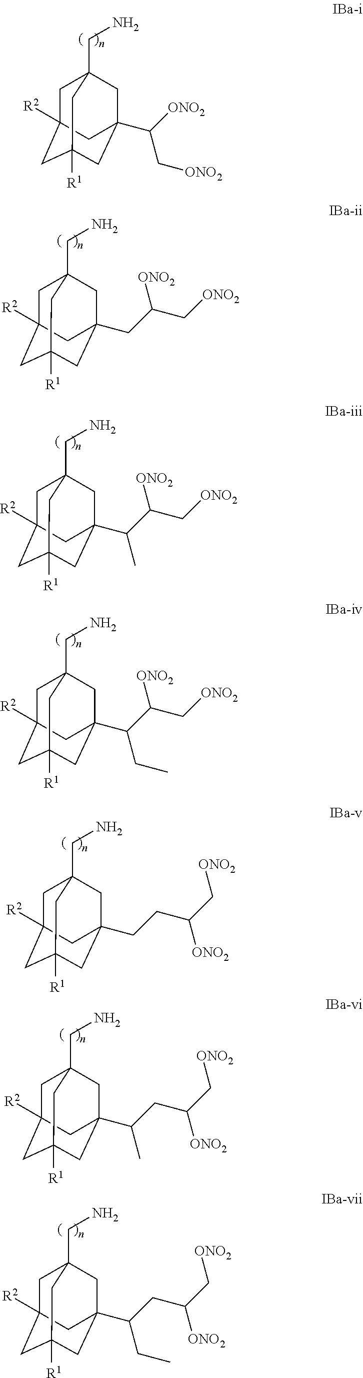

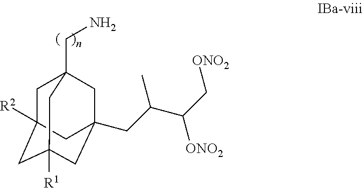

TABLE 2
For each subgenus of formula (IB-a-i) to (IB-a-vii):

| n | R¹ | R² |
|---|---|---|
| 1, 2 and 3 | methyl | methyl |
| 1, 2 and 3 | hydrogen | methyl |
| 1, 2 and 3 | methyl | hydrogen |
| 1, 2 and 3 | ethyl | ethyl |
| 1, 2 and 3 | hydrogen | ethyl |
| 1, 2 and 3 | ethyl | hydrogen |
| 1, 2 and 3 | n-propyl | n-propyl |
| 1, 2 and 3 | hydrogen | n-propyl |
| 1, 2 and 3 | n-propyl | hydrogen |
| 1, 2 and 3 | isopropyl | isopropyl |
| 1, 2 and 3 | hydrogen | isopropyl |
| 1, 2 and 3 | isopropyl | hydrogen |
| 1, 2 and 3 | n-butyl | n-butyl |
| 1, 2 and 3 | hydrogen | n-butyl |
| 1, 2 and 3 | n-butyl | hydrogen |
| 1, 2 and 3 | isobutyl | isobutyl |
| 1, 2 and 3 | hydrogen | isobutyl |
| 1, 2 and 3 | isobutyl | hydrogen |
| 1, 2 and 3 | sec-butyl | sec-butyl |
| 1, 2 and 3 | hydrogen | sec-butyl |
| 1, 2 and 3 | sec-butyl | hydrogen |
| 1, 2 and 3 | —CH₂—ONO₂ | —CH₂—ONO₂ |
| 1, 2 and 3 | hydrogen | —CH₂—ONO₂ |
| 1, 2 and 3 | —CH₂—ONO₂ | hydrogen |
| 1, 2 and 3 | —(CH₂)₂—ONO₂ | —(CH₂)₂—ONO₂ |
| 1, 2 and 3 | hydrogen | —(CH₂)₂—ONO₂ |
| 1, 2 and 3 | —(CH₂)₂—ONO₂ | hydrogen |
| 1, 2 and 3 | —(CH₂)₃—ONO₂ | —(CH₂)₃—ONO₂ |
| 1, 2 and 3 | hydrogen | —(CH₂)₃—ONO₂ |
| 1, 2 and 3 | —(CH₂)₃—ONO₂ | hydrogen |
| 1, 2 and 3 | —CH₂CH(ONO₂)CH₃ | —CH₂CH(ONO₂)CH₃ |
| 1, 2 and 3 | hydrogen | —CH₂CH(ONO₂)CH₃ |
| 1, 2 and 3 | —CH₂CH(ONO₂)CH₃ | hydrogen |
| 1, 2 and 3 | —CH₂CH(CH₃)CH₂—ONO₂ | —CH₂CH(CH₃)CH₂—ONO₂ |
| 1, 2 and 3 | hydrogen | —CH₂CH(CH₃)CH₂—ONO₂ |
| 1, 2 and 3 | —CH₂CH(CH₃)CH₂—ONO₂ | hydrogen |

In certain embodiments, the nitro-aminoadamantane compound used in the methods of the invention is further described by a subgenus of formula (IB-a-i) to (IB-a-vii), or a pharmaceutically acceptable salt thereof.

In certain embodiments, the nitro-aminoadamantane compound used in the methods of the invention is selected from:

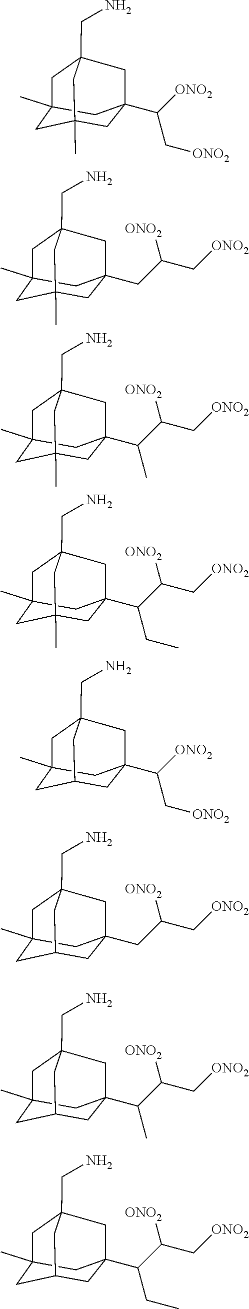

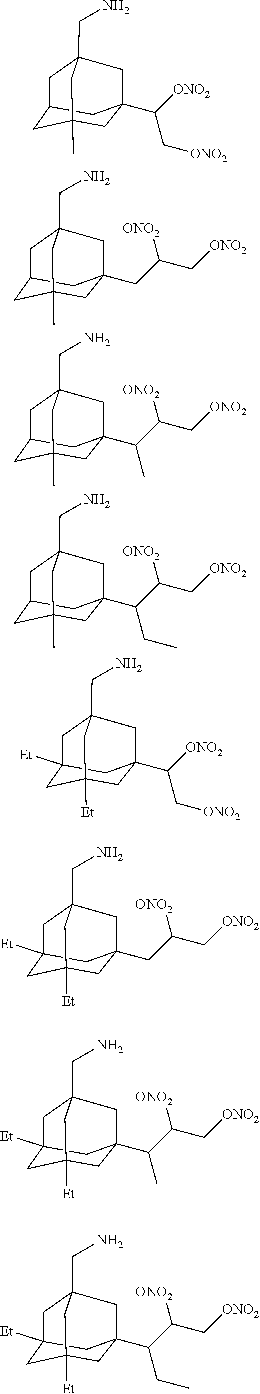

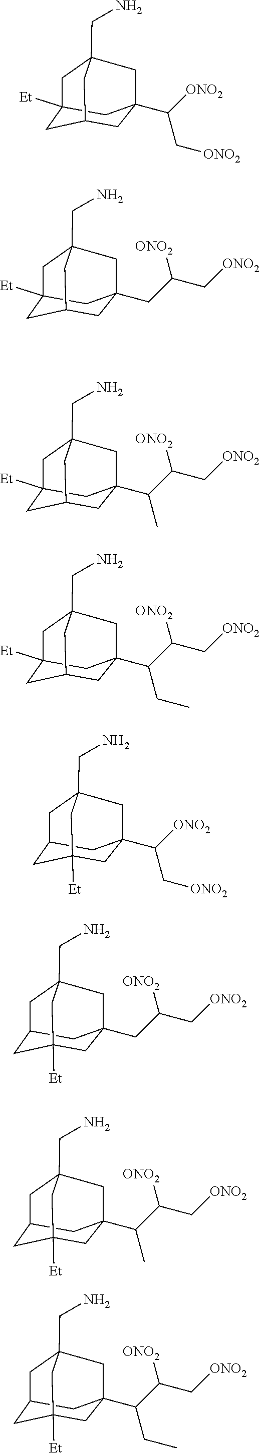

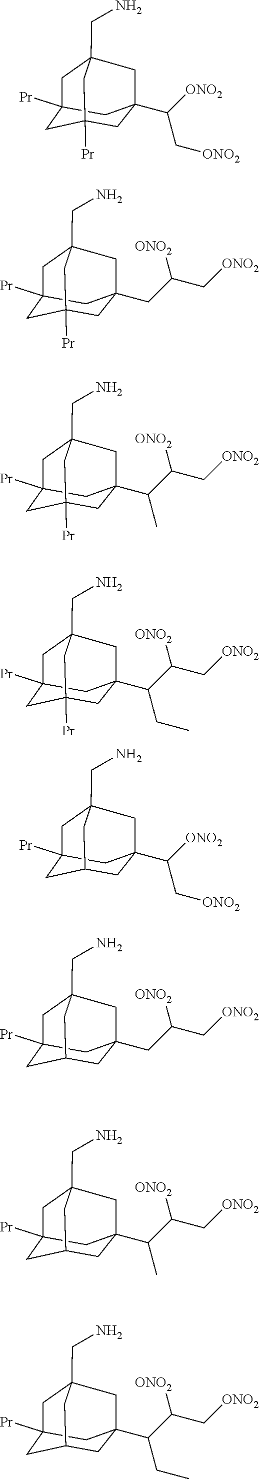

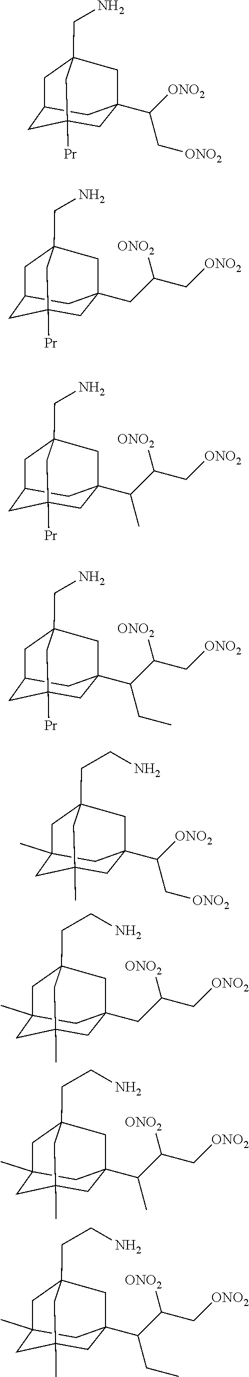

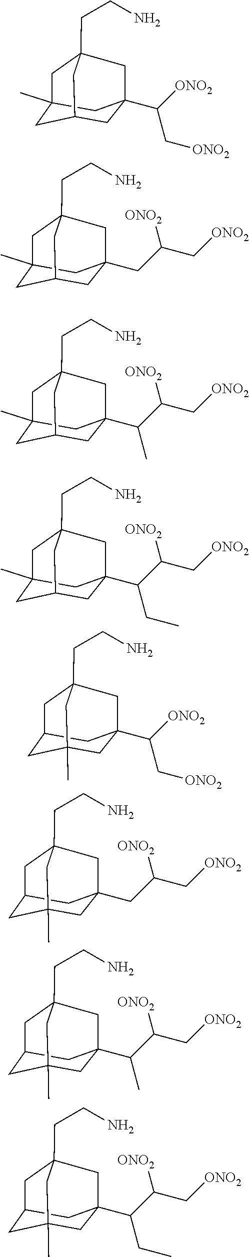

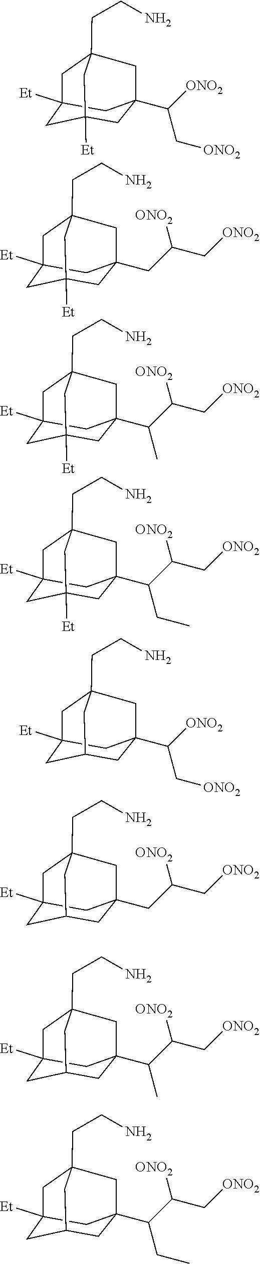

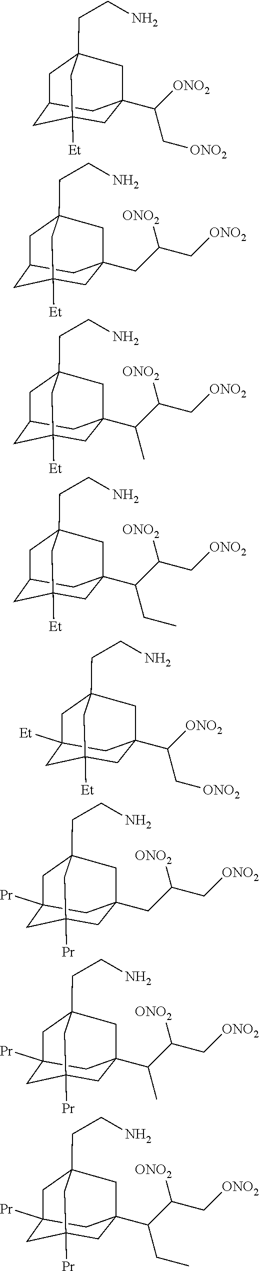

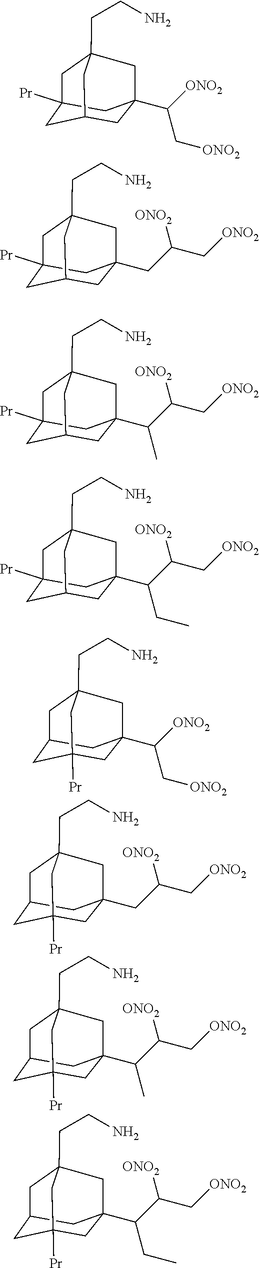

and pharmaceutically acceptable salts thereof.

Table 3 depicts representative compounds of formula (IVA-i) to (IVA-vii):

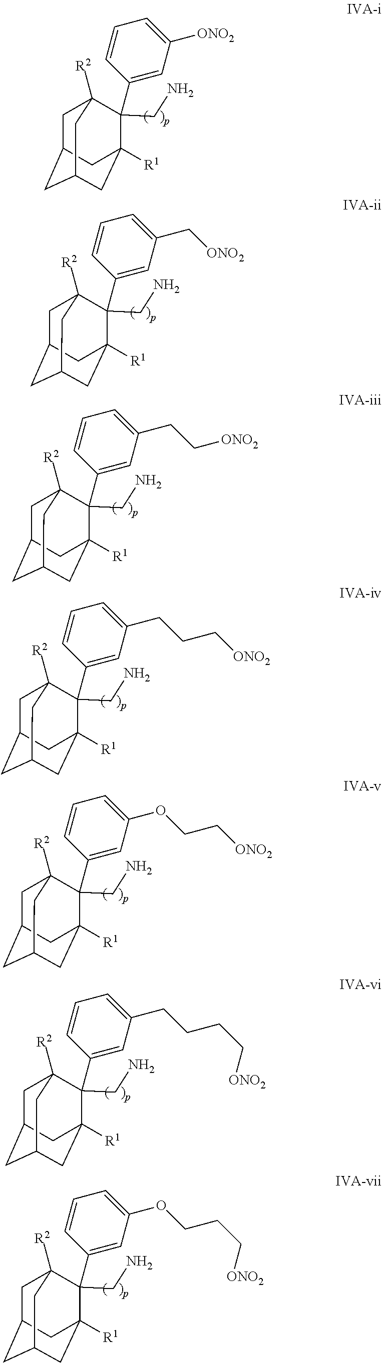

TABLE 3
For each subgenus of formula (IVA-i) to (IVA-vii)

| p | R¹ | R² |
|---|---|---|
| 0, 1, 2 and 3 | hydrogen | hydrogen |
| 0, 1, 2 and 3 | methyl | methyl |
| 0, 1, 2 and 3 | hydrogen | methyl |
| 0, 1, 2 and 3 | methyl | hydrogen |
| 0, 1, 2 and 3 | ethyl | ethyl |
| 0, 1, 2 and 3 | hydrogen | ethyl |
| 0, 1, 2 and 3 | ethyl | hydrogen |
| 0, 1, 2 and 3 | n-propyl | n-propyl |
| 0, 1, 2 and 3 | hydrogen | n-propyl |
| 0, 1, 2 and 3 | n-propyl | hydrogen |
| 0, 1, 2 and 3 | isopropyl | isopropyl |
| 0, 1, 2 and 3 | hydrogen | isopropyl |
| 0, 1, 2 and 3 | isopropyl | hydrogen |
| 0, 1, 2 and 3 | n-butyl | n-butyl |
| 0, 1, 2 and 3 | hydrogen | n-butyl |
| 0, 1, 2 and 3 | n-butyl | hydrogen |
| 0, 1, 2 and 3 | isobutyl | isobutyl |
| 0, 1, 2 and 3 | hydrogen | isobutyl |
| 0, 1, 2 and 3 | isobutyl | hydrogen |
| 0, 1, 2 and 3 | sec-butyl | sec-butyl |
| 0, 1, 2 and 3 | hydrogen | sec-butyl |
| 0, 1, 2 and 3 | sec-butyl | hydrogen |
| 0, 1, 2 and 3 | —CH₂—ONO₂ | —CH₂—ONO₂ |
| 0, 1, 2 and 3 | hydrogen | —CH₂—ONO₂ |
| 0, 1, 2 and 3 | —CH₂—ONO₂ | hydrogen |
| 0, 1, 2 and 3 | —(CH₂)₂—ONO₂ | —(CH₂)₂—ONO₂ |
| 0, 1, 2 and 3 | hydrogen | —(CH₂)₂—ONO₂ |
| 0, 1, 2 and 3 | —(CH₂)₂—ONO₂ | hydrogen |
| 0, 1, 2 and 3 | —(CH₂)₃—ONO₂ | —(CH₂)₃—ONO₂ |
| 0, 1, 2 and 3 | hydrogen | —(CH₂)₃—ONO₂ |
| 0, 1, 2 and 3 | —(CH₂)₃—ONO₂ | hydrogen |
| 0, 1, 2 and 3 | —CH₂CH(ONO₂)CH₃ | —CH₂CH(ONO₂)CH₃ |
| 0, 1, 2 and 3 | hydrogen | —CH₂CH(ONO₂)CH₃ |
| 0, 1, 2 and 3 | —CH₂CH(ONO₂)CH₃ | hydrogen |
| 0, 1, 2 and 3 | —CH₂CH(CH₃)CH₂—ONO₂ | —CH₂CH(CH₃)CH₂—ONO₂ |
| 0, 1, 2 and 3 | hydrogen | —CH₂CH(CH₃)CH₂—ONO₂ |
| 0, 1, 2 and 3 | —CH₂CH(CH₃)CH₂—ONO₂ | hydrogen |

In certain embodiments, the nitro-aminoadamantane compound used in the methods of the invention is further described by a subgenus of formula (IVA-i) to (IVA-vii), or a pharmaceutically acceptable salt thereof.

In certain embodiments, the nitro-aminoadamantane compound used in the methods of the invention is selected from:

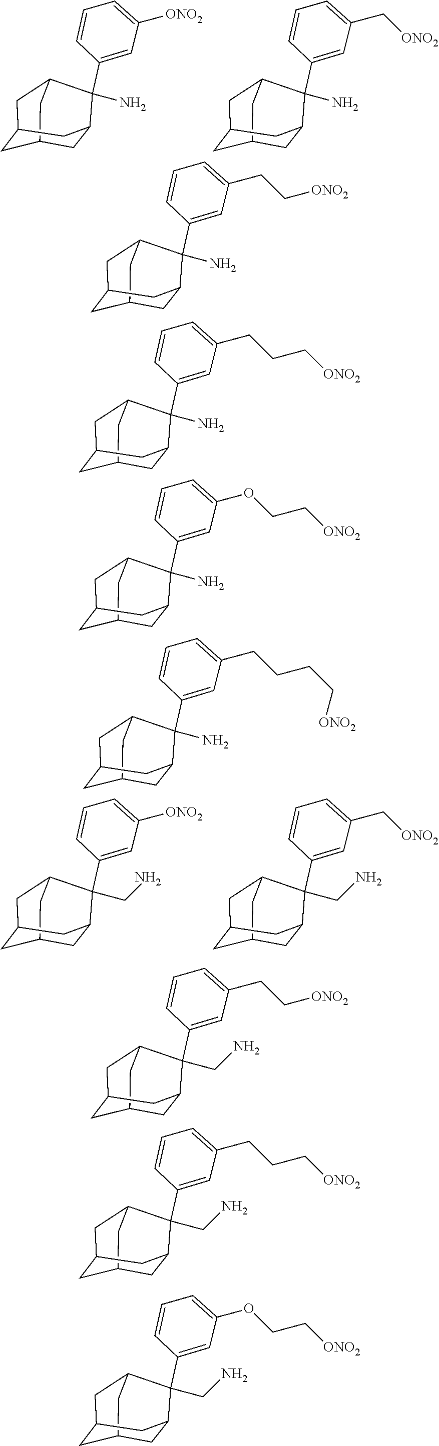

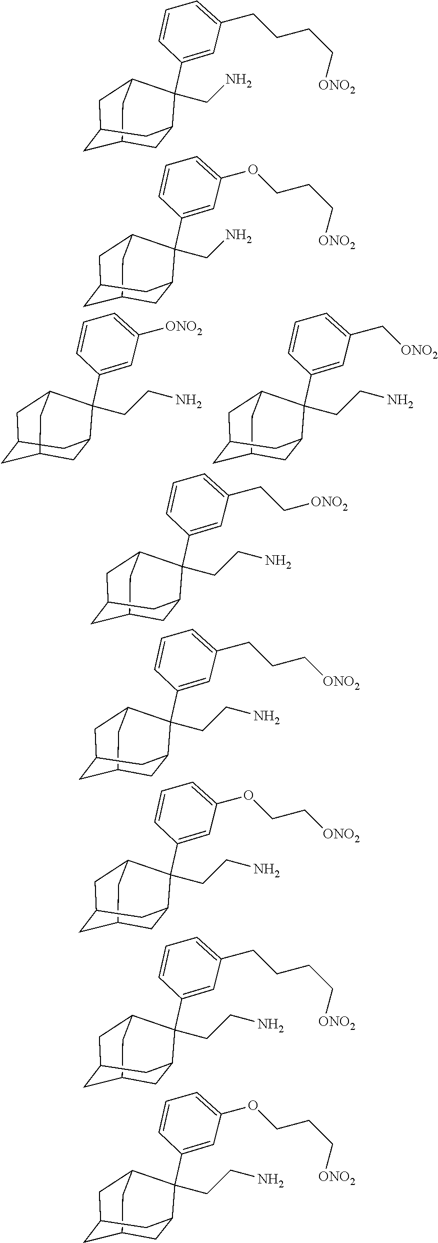

and pharmaceutically acceptable salts thereof.

Table 4 depicts representative compounds of formula (IVB-i) to (IVB-v):

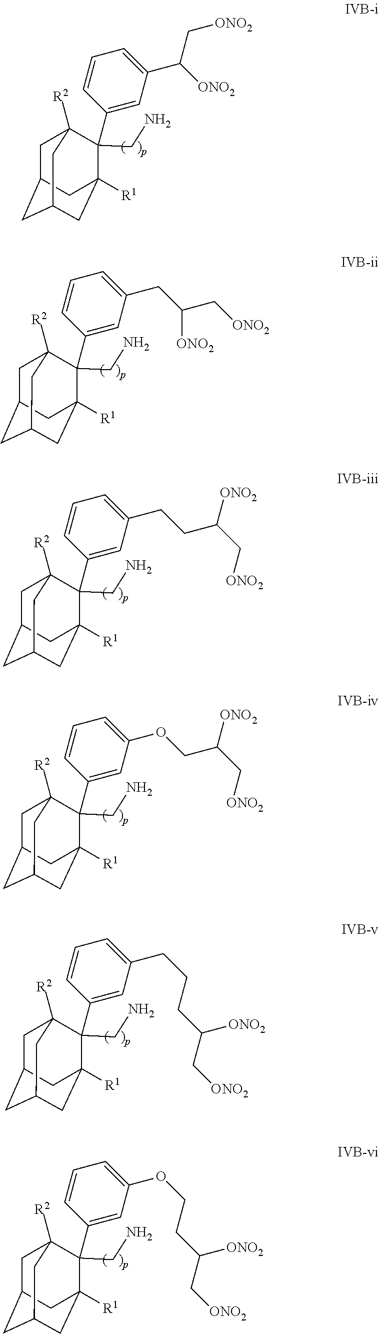

TABLE 4
For each subgenus of formula (IVB-i) to (IVB-vi)

| p | R¹ | R² |
|---|---|---|
| 0, 1, 2 and 3 | hydrogen | hydrogen |
| 0, 1, 2 and 3 | methyl | methyl |
| 0, 1, 2 and 3 | hydrogen | methyl |
| 0, 1, 2 and 3 | methyl | hydrogen |
| 0, 1, 2 and 3 | ethyl | ethyl |
| 0, 1, 2 and 3 | hydrogen | ethyl |
| 0, 1, 2 and 3 | ethyl | hydrogen |
| 0, 1, 2 and 3 | n-propyl | n-propyl |
| 0, 1, 2 and 3 | hydrogen | n-propyl |
| 0, 1, 2 and 3 | n-propyl | hydrogen |
| 0, 1, 2 and 3 | isopropyl | isopropyl |
| 0, 1, 2 and 3 | hydrogen | isopropyl |
| 0, 1, 2 and 3 | isopropyl | hydrogen |
| 0, 1, 2 and 3 | n-butyl | n-butyl |
| 0, 1, 2 and 3 | hydrogen | n-butyl |
| 0, 1, 2 and 3 | n-butyl | hydrogen |
| 0, 1, 2 and 3 | isobutyl | isobutyl |
| 0, 1, 2 and 3 | hydrogen | isobutyl |
| 0, 1, 2 and 3 | isobutyl | hydrogen |
| 0, 1, 2 and 3 | sec-butyl | sec-butyl |
| 0, 1, 2 and 3 | hydrogen | sec-butyl |
| 0, 1, 2 and 3 | sec-butyl | hydrogen |
| 0, 1, 2 and 3 | —CH₂—ONO₂ | —CH₂—ONO₂ |
| 0, 1, 2 and 3 | hydrogen | —CH₂—ONO₂ |
| 0, 1, 2 and 3 | —CH₂—ONO₂ | hydrogen |
| 0, 1, 2 and 3 | —(CH₂)₂—ONO₂ | —(CH₂)₂—ONO₂ |
| 0, 1, 2 and 3 | hydrogen | —(CH₂)₂—ONO₂ |
| 0, 1, 2 and 3 | —(CH₂)₂—ONO₂ | hydrogen |
| 0, 1, 2 and 3 | —(CH₂)₃—ONO₂ | —(CH₂)₃—ONO₂ |
| 0, 1, 2 and 3 | hydrogen | —(CH₂)₃—ONO₂ |
| 0, 1, 2 and 3 | —(CH₂)₃—ONO₂ | hydrogen |
| 0, 1, 2 and 3 | —CH₂CH(ONO₂)CH₃ | —CH₂CH(ONO₂)CH₃ |
| 0, 1, 2 and 3 | hydrogen | —CH₂CH(ONO₂)CH₃ |
| 0, 1, 2 and 3 | —CH₂CH(ONO₂)CH₃ | hydrogen |
| 0, 1, 2 and 3 | —CH₂CH(CH₃)CH₂—ONO₂ | —CH₂CH(CH₃)CH₂—ONO₂ |
| 0, 1, 2 and 3 | hydrogen | —CH₂CH(CH₃)CH₂—ONO₂ |
| 0, 1, 2 and 3 | —CH₂CH(CH₃)CH₂—ONO₂ | hydrogen |

In certain embodiments, the nitro-aminoadamantane compound used in the methods of the invention is further described by a subgenus of formula (IVB-i) to (IVB-vi), or a pharmaceutically acceptable salt thereof.

In certain embodiments, the nitro-aminoadamantane compound used in the methods of the invention is selected from:

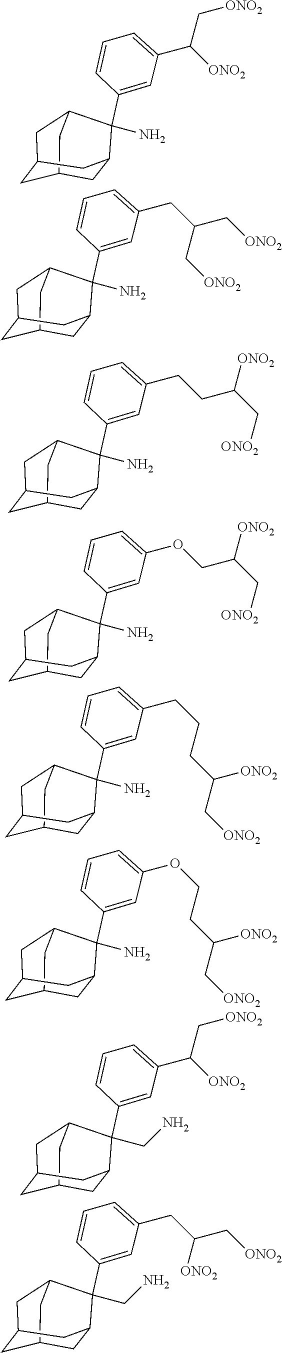

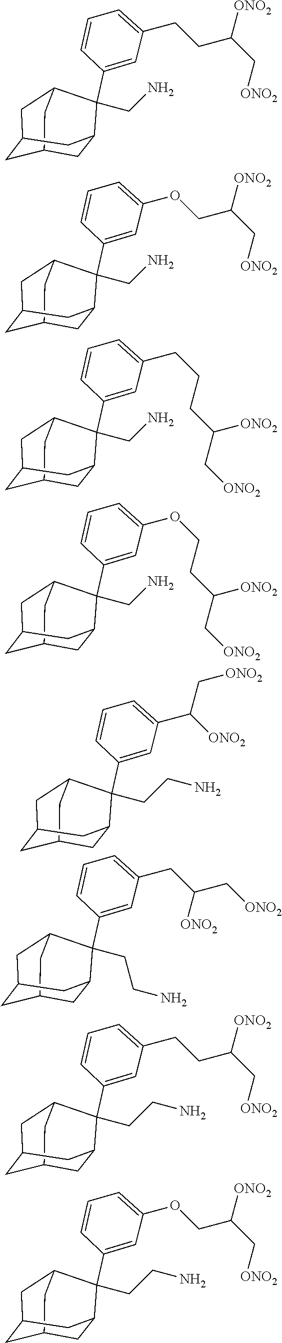

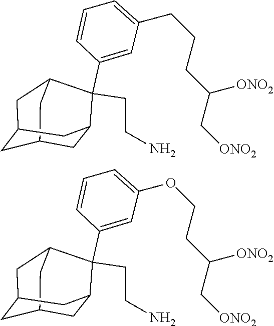

and pharmaceutically acceptable salts thereof.

In particular embodiments, instead of being an amine group, the amine group indirectly or directly connected to the C-1 or C-2 position of the nitro-aminoadamantane compounds described herein can be an amide, carbamate or urea (e.g., an —NH(C═O)R amide, —NH(C═O)OR carbamate or —NHC(═O)NR^{a}R^{b} urea group)s. In some embodiments, the —NR³R⁴ moiety of nitro-aminoadamantane compounds is —NH(C═O)R⁶, —NH(C═O)OR⁶ or —NHC(═O)NR⁷R⁸, wherein R⁶ is hydrogen (for formamide) or linear or branched C₁-C₆ alkyl, and R⁷ and R⁸ independently are hydrogen or linear or branched C₁-C₆ alkyl, or R⁷, R⁸ and the nitrogen atom to which they are attached form a 3-8-membered ring. In certain embodiments, R⁸ is hydrogen (for formamide) or linear or branched C₁-C₃ alkyl (e.g., methyl or ethyl), and R⁷ and R⁸ independently are hydrogen or linear or branched C₁-C₃ alkyl (e.g., methyl or ethyl).

Compounds of Formula (V)

The nitro-aminoadamantane compound used in the methods of the invention can be a compound of formula (V):

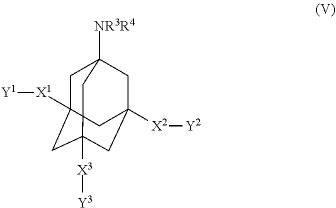

or a pharmaceutically acceptable salt thereof, wherein:

R³ and R⁴ independently are hydrogen or linear or branched C₁-C₆ alkyl, or R³, R⁴ and the nitrogen atom to which they are attached form a 3-8-membered heterocyclic ring;

R⁵ is hydrogen or linear or branched C₁-C₆ alkyl;

each of X¹, X², and X³ is, independently, selected from a bond, linear or branched -alkyl-, linear or branched -heteroalkyl-, linear or branched —O-alkyl-, linear or branched —O-heteroalkyl-, —(CH₂)ⱼ-cycloalkyl-(CH₂)ₖ—, —(CH₂)ⱼ-heterocyclyl-(CH₂)ₖ—, —(CH₂)ⱼ-aryl-(O)ₕ—(CH₂)ₖ— or —(CH₂)-heteroaryl-(O)ₕ—(CH₂)ₖ—, each of which can optionally be substituted;

each of Y¹, Y², and Y³ is, independently, selected from —ONO₂,

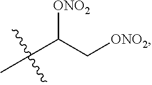

hydroxy, or a hydrogen atom, provided that at least one of Y¹, Y², and Y³ is —ONO₂ or

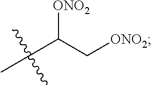

j is 0, 1, 2 or 3; k is 0, 1, 2 or 3; and h is 0 or 1.

In certain embodiments, the nitro-aminoadamantane compound used in the methods of the invention is further described by formula (VA):

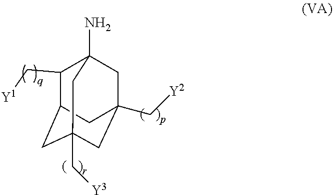

or a pharmaceutically acceptable salt thereof, wherein each of Y¹, Y², and Y³ is, independently, selected from —ONO₂, hydroxy, or a hydrogen atom; p is 0, 1, 2, 3, 4, 5 or 6; q is 0, 1, 2, 3, 4, 5 or 6; and r is 0, 1, 2, 3, 4, 5 or 6, provided that at least one of Y¹, Y², and Y³ is —ONO₂.

In certain embodiments, the nitro-aminoadamantane compound used in the methods of the invention is selected from:

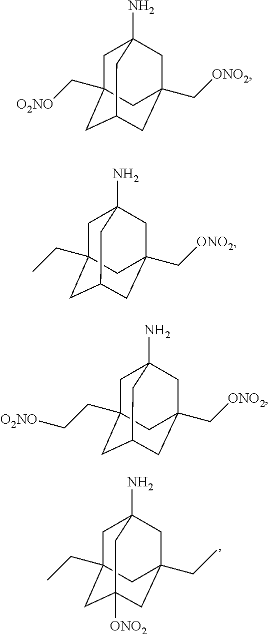

and
and pharmaceutically acceptable salts thereof.

### Dosing Regimens

The therapeutically effective amount and the frequency of administration of a nitro-aminoadamantane compound to treat or prevent a betacoronavirus infection may depend on various factors, including the type of disorder, the severity of the condition, the potency of the compound, the mode of administration, the age, body weight, general health, gender and diet of the subject, and the response of the subject to the treatment, and can be determined by the treating physician. In some embodiments, the effective dose of a nitro-aminoadamantane compound per day is from about 1, 5 or 10 mg to about 100 mg, or as deemed appropriate by the treating physician, which can be administered in a single dose or in divided doses. In certain embodiments, the effective dose of a nitro-aminoadamantane compound per day is from about 5 or 10 mg to about 50 mg or about 50-100 mg, or is about 5-10 mg, 10-20 mg, 20-30 mg, 30-40 mg, 40-50 mg, 50-60 mg, 60-70 mg, 70-80 mg, 80-90 mg, 90-100 mg, or 100-250 mg. In further embodiments, the effective dose of a nitro-aminoadamantane compound per day is about 1, 5, 10, 15, 20, 25, 30, 40, 50, 60, 70, 80, 90, 100, 125, 150, 175, 200, 225 mg, or more. In certain embodiments, the effective dose of a nitro-aminoadamantane compound per day is about 10-30 mg, or about 10, 15, 20, 25 or 30 mg.

The dosage of a nitro-aminoadamantane compound can be adjusted during the course of the treatment regimen, which can be determined by the treating physician. For example, a nitro-aminoadamantane compound can be administered in an initial daily dose for the first week of treatment, and then the daily dose of the compound can be gradually or step-wise increased for every subsequent week of treatment until a target or suitable daily maintenance dose is administered for, e.g., the fourth week of treatment and thereafter for the duration of treatment. Increasing the dose of a drug gradually or step-wise during the initial phase of treatment would allow the treating physician to determine the optimum therapeutic dose for the particular subject while avoiding or minimizing any potential side effect. For example, a first daily initial dose can be about 4 times smaller than a target daily maintenance dose and can be taken for the first week, a second daily initial dose can be about 2 times larger than the first initial dose and can be taken for the second week, a third daily initial dose can be about 3 times larger than the first initial dose and can be taken for the third week, and the target or a suitable daily maintenance dose can be taken for the fourth week and thereafter for the duration of treatment. The initial doses and the maintenance dose can be any effective dose described herein. As a non-limiting example, a first initial dose of about 5-10 mg of a nitro-aminoadamantane compound can be administered once daily for the first week, a second initial dose of about 10-20 mg can be administered once daily for the second week, a third initial dose of about 15-30 mg can be administered once daily for the third week, and a maintenance dose of about 20-40 mg can be administered once daily for the fourth week and thereafter for the duration of therapy.

Alternatively, if it is desired to establish a therapeutic level of a nitro-aminoadamantane compound quickly for the treatment of a betacoronavirus infection, a first loading dose of the compound can be administered on, e.g., day 1, an optional second loading dose can be administered on, e.g., day 2, an optional third loading dose can be administered on, e.g., day 3, and a maintenance dose of the compound can be administered daily thereafter for the duration of treatment. A loading dose can be, e.g., about 5, 4, 3, 2.5, 2 or 1.5 times larger than the maintenance dose, and the optional second and third loading doses can be, e.g., smaller than the previous loading dose. For example, relative to the maintenance dose the first loading dose can be about 4 times larger, the second loading dose can be about 3 times larger, and the third loading dose can be about 2 times larger. The maintenance dose can be any effective dose described herein. In certain embodiments, the loading dose(s) can be any effective dose described herein.

A nitro-aminoadamantane compound can be administered in any suitable frequency to treat or prevent a betacoronavirus infection, which can be determined by the treating physician. In some embodiments, a nitro-aminoadamantane compound is administered daily (including one, two, three or more times daily), once every two days, once every three days, twice weekly or once weekly, or as deemed appropriate by the treating physician. In certain embodiments, a nitro-aminoadamantane compound is administered once daily.

A nitro-aminoadamantane compound can be administered for any suitable period of time to treat a betacoronavirus infection, which can be determined by the treating physician. For the treatment of a betacoronavirus infection, in some embodiments a nitro-aminoadamantane compound is administered for a period of at least about 1 week, 2 weeks, 1 month, 3 months, or longer.

A nitro-aminoadamantane compound can be administered via any suitable route, and can be administered locally or systemically, for the treatment or prevention of a betacoronavirus infection, which can be determined by the treating physician. Potential routes of administration of a nitro-aminoadamantane compound include without limitation oral, parenteral (including intradermal, subcutaneous, intramuscular, intravascular, intravenous, intraarterial, intraperitoneal, intracavitary, intramedullary, intrathecal and topical), and topical (including dermal/epicutaneous, transdermal, mucosal, transmucosal, intranasal [e.g., by nasal spray or drop], ocular/intraocular [e.g., by eye drop], pulmonary [e.g., by oral or nasal inhalation], buccal, sublingual, rectal [e.g., by suppository], and vaginal [e.g., by suppository]). In certain embodiments, a nitro-aminoadamantane compound is administered orally (e.g., as a tablet or capsule). In other embodiments, a nitro-aminoadamantane compound is administered parenterally (e.g., intravenously, intramuscularly or subcutaneously, whether by injection or infusion).

A nitro-aminoadamantane compound may be administered to provide pre-exposure prophylaxis or after a subject has been diagnosed as having a betacoronavirus infection, or a subject exposed to a betacoronavirus. The composition may be administered, for example, for at least about 1, 2, 3, or 4 weeks pre-exposure to the betacoronavirus, or may be administered to the subject 15-30 minutes or 1, 2, 3, 4, 5, 6, 7, 8, 9, 10, 12, 20, 24, 48, or 72 hours, 2, 3, 5, or 7 days post-exposure to a betacoronavirus.

When treating a betacoronavirus infection, the nitro-aminoadamantane compound may be administered to the subject either before the occurrence of symptoms or a definitive diagnosis or after diagnosis or symptoms become evident. For example, the composition may be administered, for example, immediately after diagnosis or the clinical recognition of symptoms or 2, 4, 6, 10, 15, or 24 hours, 2, 3, 5, or 7 days after diagnosis or detection of symptoms.

In addition, single or multiple administrations of the nitro-aminoadamantane compound may be given (pre- or post-exposure and/or pre- or post-diagnosis) to a subject (e.g., one administration or administration two or more times). For example, subjects who are particularly susceptible to a betacoronavirus infection, e.g., as described herein, may require multiple treatments to establish and/or maintain protection against the virus. The dosages may then be adjusted or repeated as necessary to achieve amelioration of symptoms associated with betacoronavirus infection.

### Pharmaceutical Compostions

Additional embodiments of the disclosure relate to pharmaceutical compositions comprising a nitro-aminoadamantane compound described herein, or a pharmaceutically acceptable sat thereof, and one or more pharmaceutically acceptable excipients or carriers. The compositions can optionally contain an additional therapeutic agent. A pharmaceutical composition contains a therapeutically effective amount, or any appropriate fraction thereof, of a nitro-aminoadamantane compound, one or more pharmaceutically acceptable excipients or carriers and optionally an additional therapeutic agent, and is formulated for administration to a subject for therapeutic use.

A pharmaceutical composition contains a nitro-aminoadamantane compound and optionally an additional therapeutic agent in substantially pure form. In some embodiments, the purity of the nitro-aminoadamantane compound and the optional additional therapeutic agent independently is at least about 95%, 96%, 97%, 98% or 99%. In certain embodiments, the purity of the nitro-aminoadamantane compound and the optional additional therapeutic agent independently can be at least about 98% or 99%. In addition, a pharmaceutical composition can be substantially free of contaminants or impurities. In some embodiments, the level of contaminants or impurities other than residual solvent in a pharmaceutical composition is no more than about 5%, 4%, 3%, 2% or 1% relative to the combined weight of the intended active and inactive ingredients. In certain embodiments, the level of contaminants or impurities other than residual solvent in a pharmaceutical composition is no more than about 2% or 1% relative to the combined weight of the intended active and inactive ingredients. Pharmaceutical compositions generally are prepared according to current good manufacturing practice (GMP), as recommended or required by, e.g., the Federal Food, Drug, and Cosmetic Act § 501(a)(2)(B) and the International Conference on Harmonisation Q7 Guideline.

Pharmaceutical compositions/formulations can be prepared in sterile form. For example, pharmaceutical compositions/formulations for parenteral administration by injection or infusion generally are sterile. Sterile pharmaceutical compositions/formulations are compounded or manufactured according to pharmaceutical-grade sterilization standards known to those of skill in the art, such as those disclosed in or required by the United States Pharmacopeia Chapters 797, 1072 and 1211, and 21 Code of Federal Regulations 211.

Pharmaceutically acceptable excipients and carriers include pharmaceutically acceptable substances, materials and vehicles. Non-limiting examples of types of excipients include liquid and solid fillers, diluents, binders, lubricants, glidants, surfactants, dispersing agents, disintegration agents, emulsifying agents, wetting agents, suspending agents, thickeners, solvents, isotonic agents, buffers, pH adjusters, absorption-delaying agents, stabilizers, antioxidants, preservatives, antimicrobial agents, antibacterial agents, antifungal agents, chelating agents, adjuvants, sweetening agents, flavoring agents, coloring agents, encapsulating materials and coating materials. The use of such excipients in pharmaceutical formulations is known in the art. For example, conventional vehicles and carriers include without limitation oils (e.g., vegetable oils, such as olive oil and sesame oil), aqueous solvents {e.g., saline, buffered saline (e.g., phosphate-buffered saline [PBS]) and isotonic solutions (e.g., Ringer's solution)), and organic solvents (e.g., dimethyl sulfoxide [DMSO] and alcohols [e.g., ethanol, glycerol and propylene glycol]). Except insofar as any conventional excipient or carrier is incompatible with the active ingredient, the disclosure encompasses the use of conventional excipients and carriers in formulations containing nitro-aminoadamantane compounds. See, e.g., Remington: The Science and Practice of Pharmacy, 21st Ed., Lippincott Williams & Wilkins (Philadelphia, Pa.) (2005); Handbook of Pharmaceutical Excipients, 5th Ed., Rowe et al., Eds., The Pharmaceutical Press and the American Pharmaceutical Association (2005); Handbook of Pharmaceutical Additives, 3rd Ed., Ash and Ash, Eds., Gower Publishing Co. (2007); and Pharmaceutical Pre-formulation and Formulation, Gibson, Ed., CRC Press LLC (Boca Raton, Fla.) (2004).

Appropriate formulation can depend on various factors, such as the route of administration chosen. Potential routes of administration of pharmaceutical compositions comprising nitro-aminoadamantane compounds include without limitation oral, parenteral (including intradermal, subcutaneous, intramuscular, intravascular, intravenous, intraarterial, intraperitoneal, intracavitary, intramedullary, intrathecal and topical), and topical (including dermal/epicutaneous, transdermal, mucosal, transmucosal, intranasal [e.g., by nasal spray or drop], ocular/intraocular [e.g., by eye drop], pulmonary [e.g., by oral or nasal inhalation], buccal, sublingual, rectal [e.g., by suppository], and vaginal [e.g., by suppository]). Topical formulations can be designed to produce a local or systemic therapeutic effect.

As an example, formulations of nitro-aminoadamantane compounds suitable for oral administration can be presented as, e.g., boluses; capsules (including push-fit capsules and soft capsules), tablets, pills, cachets or lozenges; as powders or granules; as semisolids, electuaries, pastes or gels; as solutions or suspensions in an aqueous liquid or/and a non-aqueous liquid; or as oil-in-water liquid emulsions or water-in-oil liquid emulsions.

Push-fit capsules or two-piece hard gelatin capsules can contain a nitro-aminoadamantane compound in admixture with, e.g., a filler or inert solid diluent (e.g., calcium carbonate, calcium phosphate, kaolin or lactose), a binder (e.g., a starch), a glidant or lubricant (e.g., talc or magnesium stearate), and a disintegrant (e.g., crospovidone), and optionally a stabilizer or/and a preservative. For soft capsules or single-piece gelatin capsules, a nitro-aminoadamantane compound can be dissolved or suspended in a suitable liquid (e.g., liquid polyethylene glycol or an oil medium, such as a fatty oil, peanut oil, olive oil or liquid paraffin), and the liquid-filled capsules can contain one or more other liquid excipients or/and semi-solid excipients, such as a stabilizer or/and an amphiphilic agent (e.g., a fatty acid ester of glycerol, propylene glycol or sorbitol). In certain embodiments, a capsule (e.g., a hard gelatin capsule) comprises a nitro-aminoadamantane compound and sugar spheres, polyvinylpyrrolidone, hypromellose, talc, polyethylene glycol, ethylcellulose, ammonium hydroxide, oleic acid, and medium-chain triglycerides.

Tablets can contain a nitro-aminoadamantane compound in admixture with, e.g., a filler or inert diluent (e.g., calcium carbonate, calcium phosphate, lactose, mannitol or microcrystalline cellulose), a binding agent (e.g., a starch, gelatin, acacia, alginic acid or a salt thereof, or microcrystalline cellulose), a lubricating agent (e.g., stearic acid, magnesium stearate, talc or silicon dioxide), and a disintegrating agent (e.g., crospovidone, croscarmellose sodium or colloidal silica), and optionally a surfactant (e.g., sodium lauryl sulfate). The tablets can be uncoated or can be coated with, e.g., an enteric coating that protects the active ingredient from the acidic environment of the stomach, or with a material that delays disintegration and absorption of the active ingredient in the gastrointestinal (GI) tract and thereby provides a sustained action over a longer time period. In certain embodiments, a tablet comprises a nitro-aminoadamantane compound and lactose monohydrate, microcrystalline cellulose, silica colloidal anhydrous, talc and magnesium stearate, and is film-coated (e.g., a film-coating containing hypromellose, titanium dioxide and macrogol 400).

Compositions for oral administration can also be formulated as solutions or suspensions in an aqueous liquid or/and a non-aqueous liquid, or as oil-in-water liquid emulsions or water-in-oil liquid emulsions. Dispersible powder or granules of a nitro-aminoadamantane compound can be mixed with any suitable combination of an aqueous liquid, an organic solvent or/and an oil and any suitable excipients (e.g., any combination of a dispersing agent, a wetting agent, a suspending agent, an emulsifying agent or/and a preservative) to form a solution, suspension or emulsion.

Nitro-aminoadamantane compounds can also be formulated for parenteral administration by injection or infusion to circumvent GI absorption and first-pass metabolism. An exemplary parenteral route is intravenous. Additional advantages of intravenous administration include direct administration of a therapeutic agent into systemic circulation to achieve a rapid systemic effect, and the ability to administer the agent continuously or/and in a large volume if desired. Formulations for injection or infusion can be in the form of, e.g., solutions, suspensions or emulsions in oily or aqueous vehicles, and can contain excipients such as suspending agents, dispersing agents or/and stabilizing agents. For example, aqueous or non-aqueous (e.g., oily) sterile injection solutions can contain a nitro-aminoadamantane compound along with excipients such as an antioxidant, a buffer, a bacteriostat and solutes that render the formulation isotonic with the blood of the subject. Aqueous or non-aqueous sterile suspensions can contain a nitro-aminoadamantane compound along with excipients such as a suspending agent and a thickening agent, and optionally a stabilizer and an agent that increases the solubility of the nitro-aminoadamantane compound to allow for the preparation of a more concentrated solution or suspension. As another example, a sterile aqueous solution for injection or infusion (e.g., subcutaneously or intravenously) can contain a nitro-aminoadamantane compound, sodium chloride, a buffering agent (e.g., sodium citrate), a preservative (e.g., meta-cresol), and optionally a base (e.g., NaOH) or/and an acid (e.g., HCl) to adjust pH.

For topical administration, a nitro-aminoadamantane compound can be formulated as, e.g., a buccal or sublingual tablet or pill. Advantages of a buccal or sublingual tablet or pill include avoidance of GI absorption and first-pass metabolism, and rapid absorption into systemic circulation. A buccal or sublingual tablet or pill can be designed to provide faster release of the nitro-aminoadamantane compound for more rapid uptake of it into systemic circulation. In addition to a therapeutically effective amount of a nitro-aminoadamantane compound, the buccal or sublingual tablet or pill can contain suitable excipients, including without limitation any combination of fillers and diluents (e.g., mannitol and sorbitol), binding agents (e.g., sodium carbonate), wetting agents (e.g., sodium carbonate), disintegrants (e.g., crospovidone and croscarmellose sodium), lubricants (e.g., silicon dioxide [including colloidal silicon dioxide] and sodium stearyl fumarate), stabilizers (e.g., sodium bicarbonate), flavoring agents (e.g., spearmint flavor), sweetening agents (e.g., sucralose), and coloring agents (e.g., yellow iron oxide).

For topical administration, nitro-aminoadamantane compounds can also be formulated for intranasal administration. The nasal mucosa provides a big surface area, a porous endothelium, a highly vascular subepithelial layer and a high absorption rate, and hence allows for high bioavailability. An intranasal formulation can comprise a nitro-aminoadamantane compound along with excipients, such as a solubility enhancer (e.g., propylene glycol), a humectant (e.g., mannitol or sorbitol), a buffer and water, and optionally a preservative (e.g., benzalkonium chloride), a mucoadhesive agent (e.g., hydroxyethylcellulose) or/and a penetration enhancer. An intranasal solution or suspension formulation can be administered to the nasal cavity by any suitable means, including but not limited to a dropper, a pipette, or spray using, e.g., a metering atomizing spray pump. Table 5 shows exemplary excipients of nasal-spray formulations.

An additional mode of topical administration of a nitro-aminoadamantane compound is pulmonary, including by oral inhalation and nasal inhalation. The lungs serve as a portal to the systemic circulation. Advantages of pulmonary drug delivery include, for example: 1) avoidance of first-pass metabolism; 2) fast drug action; 3) large surface area of the alveolar region for absorption, high permeability of the lungs (thin air-blood barrier), and profuse vasculature of the airways; 4) reduced extracellular enzyme levels compared to the GI tract due to the large alveolar surface area; and 5) smaller doses to achieve equivalent therapeutic effect compared to other oral routes, and hence reduced systemic side effects. An advantage of oral inhalation over nasal inhalation includes deeper penetration/deposition of the drug into the lungs. Oral or nasal inhalation can be achieved by means of, e.g., a metered-dose inhaler (MDI), a dry powder inhaler (DPI) or a nebulizer, as is known in the art. In certain embodiments, a sterile aqueous solution for oral inhalation contains a nitro-aminoadamantane compound, sodium chloride, a buffering agent (e.g., sodium citrate), optionally a preservative (e.g., meta-cresol), and optionally a base (e.g., NaOH) or/and an acid (e.g., HCl) to adjust pH.

Topical formulations for application to the skin or mucosa can be useful for transdermal or transmucosal administration of a drug into the blood for systemic distribution. Advantages of topical administration can include circumvention of the GI tract (including enzymes and acid in the GI tract and absorption through it) and first-pass metabolism; delivery of a drug with a short half-life, a small therapeutic index or/and low oral bioavailability; controlled, continuous and sustained release of the drug; a more uniform plasma level or delivery profile of the drug; lower dose and less frequent dosing of the drug; reduction of systemic side effects (e.g., side effects caused by a temporary overdose or an overly high peak plasma drug concentration); minimal or no invasiveness; ease of self-administration; and increased patient compliance.

In general, compositions suitable for topical administration include without limitation liquid or semi-liquid preparations such as sprays, gels, liniments, lotions, oil-in-water or water-in-oil emulsions such as creams, foams, ointments and pastes, and solutions or suspensions such as drops (e.g., eye drops, nose drops and ear drops). Various excipients can be included in a topical formulation. For example, solvents, including a suitable amount of an alcohol, can be used to solubilize the drug. Other optional excipients include without limitation gelling agents, thickening agents, emulsifiers, surfactants, buffers, stabilizers, antioxidants, preservatives, cooling agents, opacifiers, colorants and fragrances. For a drug having a low rate of permeation through the skin or mucosal tissue, a topical formulation can contain a chemical permeation enhancer (CPE, such as a surfactant) to increase permeation of the drug through the skin or mucosal tissue. A topical formulation can also contain an irritation-mitigating excipient that reduces any irritation to the skin or mucosa caused by the drug, the CPE or any other component of the formulation.

In some embodiments, a topical composition comprises a nitro-aminoadamantane compound dissolved, dispersed or suspended in a carrier. The carrier can be in the form of, e.g., a solution, a suspension, an emulsion, an ointment or a gel base, and can contain, e.g., petrolatum, lanolin, a wax (e.g., bee wax), mineral oil, a long-chain alcohol, polyethylene glycol or polypropylene glycol, a diluent (e.g., water or/and an alcohol [e.g., ethanol or propylene glycol]), a gel, an emulsifier, a thickening agent, a stabilizer or a preservative, or any combination thereof.

A topical composition can include, or a topical formulation can be administered by means of, e.g., a transdermal or transmucosal delivery device, such as a transdermal patch, a microneedle patch or an iontophoresis device. A topical composition can deliver a nitro-aminoadamantane compound transdermally or transmucosally via a concentration gradient (with or without the use of a CPE) or an active mechanism (e.g., iontophoresis or microneedles).

In some embodiments, a nitro-aminoadamantane compound is administered via a transdermal patch. In certain embodiments, a transdermal patch is a reservoir-type patch comprising an impermeable backing layer/film, a liquid- or gel-based drug reservoir, a semi-permeable membrane that serves as a rate-limiting or rate-controlling diffusion barrier, and a skin-contacting adhesive layer. The semi-permeable membrane can be composed of, e.g., a suitable polymeric material such as cellulose nitrate or acetate, polyisobutene, polypropylene, polyvinyl acetate or a polycarbonate. In other embodiments, a transdermal patch is a drug-in-adhesive patch comprising an impermeable backing layer/film and a skin-contacting adhesive layer incorporating the drug in a polymeric or viscous adhesive. The adhesive of the drug-loaded, skin-contacting adhesive layer can be, e.g., a pressure-sensitive adhesive (PSA), such as a PSA composed of an acrylic polymer (e.g., polyacrylate), a polyalkylene (e.g., polyisobutylene) or a silicone-based polymer (e.g., silicone-2675 or silicone-2920). Transdermal drug-delivery systems, including patches, can be designed to provide controlled and prolonged release of a drug over a period of about 1 week, 2 weeks, 3 weeks, 1 month or longer.

In some embodiments, a nitro-aminoadamantane compound is delivered from a sustained-release composition. As used herein, the term “sustained-release composition” encompasses sustained-release, prolonged-release, extended-release, delayed-release, slow-release and controlled-release compositions, systems and devices. Advantages of a sustained-release composition include without imitation a more uniform blood level of the drug (e.g., avoidance of wide peak-to-trough fluctuations), delivery of a therapeutically effective amount of the drug over a prolonged time period, reduced frequency of administration, and reduced side effects (e.g., avoidance of a drug overdose). In certain embodiments, the sustained-release composition delivers the nitro-aminoadamantane compound over a period of at least about 1 day, 2 days, 3 days, 1 week, 2 weeks, 3 weeks, 1 month, 2 months, 3 months or longer.

In some embodiments, the sustained-release composition is a drug-encapsulation system, such as nanoparticles, microparticles or a capsule made of, e.g., a biodegradable polymer or/and a hydrogel. In certain embodiments, the sustained-release composition comprises a hydrogel. Non-limiting examples of polymers of which a hydrogel can be composed include polyvinyl alcohol, acrylate polymers (e.g., sodium polyacrylate), and other homopolymers and copolymers having a relatively large number of hydrophilic groups (e.g., hydroxyl or/and carboxylate groups). In other embodiments, the sustained-release drug-encapsulation system comprises a membrane-enclosed reservoir, wherein the reservoir contains a drug and the membrane is permeable to the drug. Such a drug-delivery system can be in the form of, e.g., a transdermal patch.

In certain embodiments, the sustained-release composition is formulated as polymeric nanoparticles or microparticles, wherein the polymeric particles can be delivered, e.g., by injection or from an implant. In some embodiments, the polymeric implant or polymeric nanoparticles or microparticles are composed of a biodegradable polymer. In certain embodiments, the biodegradable polymer comprises lactic acid or/and glycolic acid [e.g., an L-lactic acid-based copolymer, such as poly(L-lactide-co-glycolide) or poly(L-lactic acid-co-D,L-2-hydroxyoctanoic acid)]. For example, biodegradable polymeric microspheres composed of polylactic acid or/and polyglycolic acid can serve as sustained-release pulmonary drug-delivery systems. The biodegradable polymer of the polymeric implant or polymeric nanoparticles or microparticles can be selected so that the polymer substantially completely degrades around the time the period of treatment is expected to end, and so that the byproducts of the polymer's degradation, like the polymer, are biocompatible.

In further embodiments, a sustained-release composition comprises a dendrimer. In certain embodiments, the dendrimer is a water-soluble dendrimer, such as a poly(amidoamine) (PAMAM) dendrimer. In some embodiments, a dendrimer encapsulates a drug through the formation of a dendrimer-drug supramolecular assembly. In other embodiments, a sustained-release composition comprises a water-soluble polymer [e.g., poly(DL-lactide)] or a liposome encapsulating a drug complexed with a dendrimer.

In other embodiments, the sustained-release composition is an oral dosage form, such as a tablet or capsule. For example, a drug can be embedded in an insoluble porous matrix such that the dissolving drug must make its way out of the matrix before it can be absorbed through the GI tract. Alternatively, a drug can be embedded in a matrix that swells to form a gel through which the drug exits. Sustained release can also be achieved by way of a single-layer or multi-layer osmotic controlled-release oral delivery system (OROS). An OROS is a tablet with a semi-permeable outer membrane and one or more small laser-drilled holes in it. As the tablet passes through the body, water is absorbed through the semi-permeable membrane via osmosis, and the resulting osmotic pressure pushes the drug out through the hole(s) in the tablet and into the GI tract where it can be absorbed.

For a delayed or sustained release of a nitro-aminoadamantane compound, a composition can also be formulated as, e.g., a depot that can be implanted in or injected into a subject, e.g., intramuscularly or subcutaneously. A depot formulation can be designed to deliver the nitro-aminoadamantane compound over an extended period of time, e.g., over a period of at least about 1 week, 2 weeks, 3 weeks, 1 month, 6 weeks, 2 months, or longer. For example, a nitro-aminoadamantane compound can be formulated with a polymeric material (e.g., polyethylene glycol [PEG], polylactic acid [PLA] or polyglycolic acid [PGA], or a copolymer thereof [e.g., PLGA]), a hydrophobic material (e.g., as an emulsion in an oil) or/and an ion-exchange resin, or as a sparingly soluble derivative (e.g., a sparingly soluble salt). As an illustrative example, a nitro-aminoadamantane compound can be incorporated or embedded in sustained-release microparticles composed of PLGA and formulated as a monthly depot.

A nitro-aminoadamantane compound can also be contained or dispersed in a matrix material. The matrix material can comprise a polymer (e.g., ethylene-vinyl acetate) and controls the release of the compound by controlling dissolution or/and diffusion of the compound from, e.g., a reservoir, and can enhance the stability of the compound while contained in the reservoir. Such a “release system” can be designed as a sustained-release system, can be configured as, e.g., a transdermal or transmucosal patch, and can contain an excipient that can accelerate the compound's release, such as a water-swellable material (e.g., a hydrogel) that aids in expelling the compound out of the reservoir. U.S. Pat. Nos. 4,144,317 and 5,797,898 describe examples of such a release system.

The release system can provide a temporally modulated release profile (e.g., pulsatile release) when time variation in plasma levels is desired, or a more continuous or consistent release profile when a constant plasma level is desired. Pulsatile release can be achieved from an individual reservoir or from a plurality of reservoirs. For example, where each reservoir provides a single pulse, multiple pulses (“pulsatile” release) are achieved by temporally staggering the single pulse release from each of multiple reservoirs. Alternatively, multiple pulses can be achieved from a single reservoir by incorporating several layers of a release system and other materials into a single reservoir. Continuous release can be achieved by incorporating a release system that degrades, dissolves, or allows diffusion of a compound through it over an extended time period. In addition, continuous release can be approximated by releasing several pulses of a compound in rapid succession (“digital” release). An active release system can be used alone or in conjunction with a passive release system, as described in U.S. Pat. No. 5,797,898.

In addition, pharmaceutical compositions comprising a nitro-aminoadamantane compound can be formulated as, e.g., liposomes, micelles (e.g., those composed of biodegradable natural or/and synthetic polymers, such as lactosomes), nanoparticles (e.g., lipid nanoparticles such as solid lipid nanoparticles), microparticles or microspheres, whether or not designed for sustained release.

For example, liposomes can be used as sustained-release pulmonary drug-delivery systems that deliver a drug to the alveolar surface and then the circulation. As another example, lipid nanoparticles containing a lipophilic drug can be delivered into the lungs and then the circulation by oral inhalation.

In some embodiments, liposomes or micelles are composed of one or more phospholipids. Phospholipids include without limitation phosphatidic acids (e.g., DEPA, DLPA, DMPA, DOPA, DPPA and DSPA), phosphatidylcholines (e.g., DDPC, DEPC, DLPC, DLOPC, DMPC, DOPC, DPPC, DSPC, MPPC, MSPC, PLPC, PMPC, POPC, PSPC, SMPC, SOPC and SPPC), phosphatidylethanolamines (e.g., DEPE, DLPE, DMPE, DOPE, DPPE, DSPE and POPE), phosphatidylglycerols (e.g., DEPG, DLPG, DMPG, DOPG, DPPG, DSPG and POPG), phosphatidylserines (e.g., DLPS, DMPS, DOPS, DPPS and DSPS), and salts (e.g., sodium and ammonium salts) thereof. In certain embodiments, liposomes or micelles are composed of one or more phosphatidylcholines. Liposomes have a hydrophilic core, so liposomes are particularly suited for delivery of hydrophilic drugs, whereas micelles have a hydrophobic core, so micelles are particularly suited for delivery of hydrophobic drugs. The salt group of a salt form of a nitro-aminoadamantane compound provides hydrophilicity, while the adamantyl scaffold provides hydrophobicity. Liposomes and micelles can permeate across biological membranes. Liposomes and micelles can provide sustained release of a drug based in part on the rate of degradation of the liposomes and micelles.

The pharmaceutical compositions can be manufactured in any suitable manner known in the art, e.g., by means of conventional mixing, dissolving, suspending, granulating, dragee-making, levigating, emulsifying, encapsulating, entrapping or compressing processes.

The compositions can be presented in unit dosage form as a single dose wherein all active and inactive ingredients are combined in a suitable system, and components do not need to be mixed to form the composition to be administered. A unit dosage form generally contains a therapeutically effective dose of the drug, but can contain an appropriate fraction thereof. A representative example of a unit dosage form is a tablet, capsule or pill for oral uptake.

Alternatively, the compositions can be presented as a kit, wherein the active ingredient, excipients and carriers (e.g., solvents) are provided in two or more separate containers (e.g., ampules, vials, tubes, bottles or syringes) and need to be combined to form the composition to be administered. The kit can contain instructions for storing, preparing and administering the composition (e.g., a solution to be injected intravenously).

A kit can contain all active and inactive ingredients in unit dosage form or the active ingredient and inactive ingredients in two or more separate containers, and can contain instructions for administering or using the pharmaceutical composition to treat a betacoronavirus infection.

In some embodiments, a kit contains a nitro-aminoadamantane compound or a pharmaceutically acceptable salt thereof, or a pharmaceutical composition comprising the same, and instructions for administering the compound or the pharmaceutical composition to treat or prevent a betacoronavirus infection.

### EXAMPLES

The following examples are put forth to provide those of ordinary skill in the art with a description of how the compositions and methods described herein may be used, made, and evaluated, and are intended to be purely exemplary of the invention and are not intended to limit the scope of what the inventors regard as their invention.

### Example 1. Effect of Nitro-Aminoadamantane Compounds on SARS-CoV-2 Replication

Vero E6 cells are grown in 24-well plates and infected with SARS-CoV-2 at an MOI (multiplicity of infection) of 1. Cells are treated with a nitro-aminoadamantane compound either 1 h before infection and 1 h, 3 h or 6 h post-infection or only 1 h, 3 h, 6 h post-infection without pretreatment. At 24 h post-infection, cells are harvested, and total RNA was extracted using an RNeasy mini kit (Qiagen). Viral RNA is quantitated to assess the extent of inhibition in the presence of the nitro-aminoadamantane compound.

### Example 2. Effect of Nitro-Aminoadamantane Compounds on In Vivo Coronavirus Infection

Six- to eight-week-old C57BL/6 mice are injected with (10 mg/kg) nitro-aminoadamantane or vehicle 30 minutes prior to infection with mouse hepatitis coronavirus strain A59 (MHV-A59). Treatment with nitro-aminoadamantane or vehicle is repeated at day 2. (A) Each of the following days the body weights of the animals are measured until the animals are euthanized at day 4. The body weights are expressed as percentages relative to the weights at the beginning of the experiment (day 0 100%). (B) The livers are isolated and homogenized. The amount of infectious virus in the homogenates is determined by plaque assays. The virus titers are expressed as PFU/g of tissue (the threshold of detection is 10 PFU/g). (C) The relative amounts of viral genomic RNA in the liver homogenates are determined by quantitative TaqMan RT-PCR on the 1b region of the MHV genome.

### Example 3. Effect of Nitro-Aminoadamantane Compounds in Subjects Suffering from SARS-CoV-2 Infections

Subjects identified as being infected with SARS-CoV-2, the virus that causes COVID-19, are treated with oral doses (75 mg BID) of compound A (structure below) for a period of two to four weeks, and monitored for up to 2 months to assess the incidents of pneumonitis, pneumonia, acute respiratory distress syndrome, pneumonia, respiratory failure, septic shock, organ failure, cytokine storm, and death.

Compound A is selected from:

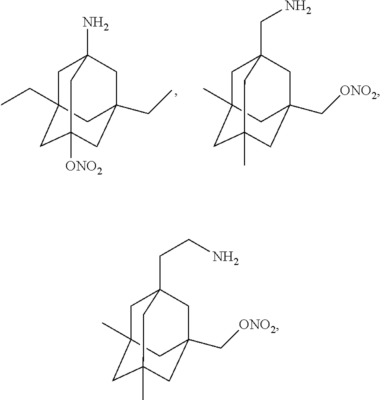

or a pharmaceutically acceptable salt thereof.

At the end of the study, subjects infected with SARS-CoV-2 receiving treatment with compound A can experience a lower risk of sever COVID-19 outcomes, including a lower risk of pneumonitis, pneumonia, acute respiratory distress syndrome, pneumonia, respiratory failure, septic shock, organ failure, cytokine storm, and/or death, in comparison to untreated subjects infected with SARS-CoV-2. Furthermore, the risk of hospitalization or the duration of hospitalization can be reduced in the subjects treated with compound A.

### Example 4. Effect of Nitro-Aminoadamantane Compounds in Subjects at Risk of Contracting SARS-CoV-2 Infections

Subjects identified as being at risk SARS-CoV-2, the virus that causes COVID-19, are treated with oral doses (75 mg BID) of compound A (structure below) for a period of one to 12 months, and monitored for up to 12 months to assess the incidents of pneumonitis, pneumonia, acute respiratory distress syndrome, pneumonia, respiratory failure, septic shock, organ failure, cytokine storm, and death.

Compound A is selected from:

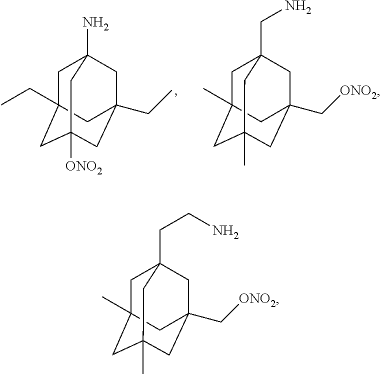

or a pharmaceutically acceptable salt thereof.

At the end of the study, subjects at risk of contracting SARS-CoV-2 receiving treatment with compound A can experience a lower infection rate and a lower risk of sever COVID-19 outcomes, including a lower risk of pneumonitis, pneumonia, acute respiratory distress syndrome, pneumonia, respiratory failure, septic shock, organ failure, cytokine storm, and/or death, in comparison to untreated subjects at risk of infection. Furthermore, the risk of hospitalization or the duration of hospitalization can be reduced in the subjects treated with compound A.

### Example 5. Dual-Mechanism Nitro-Aminoadamantane Compounds Inhibiting Viral Entry and Propagation

Prevention of infection and propagation of SARS-CoV-2 is of high priority in the COVID-19 pandemic. Here, we describe S-nitrosylation of multiple proteins involved in SARS-CoV-2 infection, including angiotensin converting enzyme 2 (ACE2), the receptor for viral entry. This reaction prevents binding of ACE2 to the SARS-CoV-2 spike protein, thereby inhibiting viral entry, infectivity, and cytotoxicity. Aminoadamantane compounds also inhibit coronavirus ion channels formed by envelope (E) protein. Accordingly, nitro-aminoadamantane compounds can act via a dual-mechanism that inhibits viral entry by S-nitrosylating ACE2, and viral propagation via E-protein channel blockade. As shown in the Examples, these non-toxic compounds are active in vitro and in vivo in the Syrian hamster COVID-19 model, and thus provide a novel potential avenue for therapy.

Methods:

Cell Lines

HEK293T (System Biosciences, LV900A-1) cells were maintained in Dulbecco's modified Eagle's medium (DMEM) with GlutaMAX™ (DMEM Media-GlutaMAX™-I, Life Technologies, 10566016) supplemented with 10% fetal bovine serum (FBS; Sigma, F7524), 100 IU/ml, and 100 μg/ml penicillin-streptomycin (Thermo Fisher Scientific, 10378016) at 37° C. in a 5% CO₂ incubator. Transfections were carried out with Lipofectamine 2000 (Life Technologies, 11688019) using the protocol recommended by the manufacturer. HeLa-ACE2 cells.

Plasmids

Studies were performed using hACE2 plasmids (Addgene plasmid #1786; http://n2t.net/addgene:1786; RRID:Addgene_1786) was. The C262A, C498A, C261/498A mutant ACE2 constructs were generated using the QuikChange Lightning Multi Site-Directed Mutagenesis Kit (Agilent Technologies, 210514) according to the manufacturer's protocol. Studies were performed using pGBW-m4252984 (SARS-CoV-2 E [envelope]) (Addgene plasmid #153898). MLV-gag/pol, MLV-CMV-Luciferase, SARS-CoV-2, and VSV-G plasmids.

Biotin-Switch Assays and Immunoblots

For analysis of S-nitrosylated proteins, we performed the biotin-switch assay as previously described (see Cho et al., Science 324, 102-105 (2009); Nakamura et al., Mol. Cell 39, 184-195 (2010); and Uehara et al., Nature 441, 513-517 (2006)). In brief, cells or tissue samples were lysed with HENTS buffer (100 mM Hepes, pH 7.4, 1 mM EDTA, 0.1 mM neocuproine, 0.1% SDS, and 1% Triton X-100) containing 10 mM methyl methanethiosulfonate [MMTS]). SDS solution (2% w/v) was added to lysed samples to a final concentration of 1% and incubated for 20 minutes at 45° C. with frequent vortexing to facilitate blockade of free thiol groups. After removing excess MMTS by acetone precipitation, S-nitrosothiols were reduced to thiols with 20 mM ascorbate. Newly formed thiols were then linked with the sulfhydryl-specific biotinylating reagent N-[6-biotinamido]-hexyl]-I′-(2′pyridydithio) propionamide (Biotin-HPDP; Dojindo, SB17-10). Unreacted biotin-HPDP was removed by acetone precipitation, and the pellet was resuspended in HENS buffer (100 mM Hepes, pH 7.4, 1 mM EDTA, 0.1 mM neocuproine, 1% SDS), neutralized, and centrifuged to clear any undissolved debris. Five percent of the supernatant was used as the input for the loading control. Biotinylated proteins were pulled down with High Capacity NeutrAvidin-Agarose beads (Thermo Scientific, 29202) and analyzed by immunoblotting for S-nitrosylated ACE2, TMPRSS2, or spike (S) protein. Protein samples were subjected to Bolt Bis-Tris Plus (Thermo Fisher Scientific, NW00102BOX) gel electrophoresis and transferred to PVDF membranes (Millipore, IPFL00010). Membranes were blocked with Odyssey blocking buffer (Li-Cor, 927-40000) for 30 minutes at RT and then probed with primary antibodies against ACE2 (1:3000, Abcam, ab15348; 1:3000, Proteintech, 21115-1-AP), TMPRSS2 (1:1000, Santa Cruz, sc-515727), or SARS-CoV-2 spike protein (1:2000, Abcam, ab275759). After incubation with secondary antibodies (IR-dye 680LT-conjugated goat anti-mouse [1:20,000; Li-Cor, 926-68020] or IR-dye 800CW-conjugated goat anti-rabbit [1:15,000; Li-Cor, 926-32211]), membranes were scanned with an Odyssey infrared imaging system (Li-Cor). Image Studio (Li-Cor) software was used for densitometric analysis of immunoblots.

Immunocytochemistry for SARS-CoV-2 Spike Protein

Purified recombinant SARS-CoV-2 spike (S1+S2) protein (10 μg/ml, Sino Biological, 40589-V08B1) exposed cells were fixed with 4% PFA for 15 minutes at RT, washed 3 times with PBS, and blocked (3% BSA, 0.3% Triton X-100 in PBS) for 30 minutes at RT. Cells were incubated with anti-SARS-CoV-2 spike protein antibodies (1:200, Sino Biological, 40150-R007) overnight at 4° C., followed by incubation with Alexa Fluor 488-conjugated secondary antibody. Cells were counterstained with 1 μg/ml Hoechst dye 33342 (Invitrogen). Cell images were acquired with a Nikon A1 Confocal Microscope using a 20×10.75 air objective (1 μm Z-stack). Maximum intensity projection of images was generated, and fluorescence intensity was analyzed.

Co-Immunoprecipitation Experiments

Cultured cells were harvested and lysed in 1% Triton X-100 in PBS. Equivalent protein quantities were immunoprecipitated with anti-ACE2 antibody (Abcam, ab15348)-conjugated magnetic beads (Dynabeads™ Protein A; Thermo Fisher Scientific, 10002D) for 90 minutes at RT. Immunoprecipitants were eluted and subjected to immunoblotting with anti-ACE2 antibody (1:1000, Cell Signaling, #15983) and anti-SARS-CoV-2 spike protein antibody (1:2000, Abcam, ab275759).

Mass Spectrometry Analysis of S-Nitrosylated ACE2 Protein

Biotin switch was performed as described above. Biotinylated proteins were then precipitated with iced acetone, pelleted, and solubilized in HENS buffer (100 mM Hepes, pH 7.4, 1 mM EDTA, 0.1 mM neocuproine, 1% SDS). The samples were desalted using a ZebaSpin desalting column (Thermo Scientific) pre-equilibrated with PBS, and biotinylated ACE2 protein was immunoprecipitated as described above. Immunoprecipitated ACE2 was eluted in 1% SDS solution and precipitated using methanol-chloroform. Dried pellets were dissolved in 8 M urea/100 mM triethylammonium bicarbonate (TEAB, pH 8.5). Samples were diluted to 2 M urea/100 mM TEAB, and proteins were trypsin digested overnight at 37° C. The digested ACE2 peptides were enriched a second time by biotin-avidin affinity to enrich biotinylated peptides representing the initial SNO sites. After avidin enrichment, peptides were eluted by reduction using tris(2-carboxyethyl)phosphine (TCEP).

Samples were analyzed on a Thermo Orbitrap Eclipse mass spectrometer (Thermo). Samples were injected directly onto a 25 cm, 100 μm ID column packed with ethylene bridged hybrid (BEH) 1.7 μm C18 resin (Waters). Samples were separated at a flow rate of 300 nl/min on a nLC 1200 (Thermo) using a gradient of solution A (0.1% formic acid in water, 5% acetonitrile) and solution B (80% acetonitrile/0.1% formic acid). Specifically, a gradient of 1-25% B over 100 min, an increase to 40% B over 20 min, an increase to 100% B over another 10 min and held at 90% B for a 10 min was used for a 140 min total run time. Peptides were eluted directly from the tip of the column and nanosprayed directly into the mass spectrometer by application of 2.5 kV voltage at the back of the column. The Orbitrap Eclipse mass spectrometer was operated in data dependent mode. Full MS1 scans were collected in the Orbitrap at 120k resolution. The cycle time was set to 3 s, and within this 3 s, the most abundant ions per scan were selected for high energy collisional dissociation (HCD) with detection in the Orbitrap. Monoisotopic precursor selection was enabled and dynamic exclusion was used with exclusion duration of 5 s.

Protein and peptide identification were done with Integrated Proteomics Pipeline—IP2 (Integrated Proteomics Applications). Tandem mass spectra were extracted from raw files using RawConverter⁴⁵ and searched with ProLuCID against Uniprot human database. The search space included all fully-tryptic and half-tryptic peptide candidates. Data were searched with 50 ppm precursor ion tolerance and 600 ppm fragment ion tolerance. Identified proteins were filtered to 10 ppm precursor ion tolerance using DTASelect utilizing a target-decoy database search strategy to control the false discovery rate to 1% at the protein level.

Molecular Dynamics Simulations

The fullyglycosylated, Cys²⁶¹/Cys⁴⁹⁸-S-nitrosylated model of human ACE2 dimer bound to two SARS-CoV-2 spike's receptor binding domains (RBD) (with a 1:1 stoichiometry) is based on the cryo-EM structure of the ACE2/RBD/B0AT1 complex (PDB ID: 6M17). BOAT1 dimer chaperone coordinates were manually removed and N-glycans were added on both ACE2 and RBD. The side chain of Cys²⁶¹ and Cys⁴⁹⁶ was S-nitrosylated in both ACE2 protomers using Schrödinger Maestro (Schrödinger Release 2020-4: Maestro, Schrödinger, LLC, New York, N.Y., 2020.). ParamChem web interface was used to generate CHARMM36 suitable parameters for the S—N═O moiety. The glycosylated and S-nitrosylated ACE2/RBD construct was inserted into a lipid bilayer patch of 350 Å×350 Å with a composition similar to that of mammalian cell membranes (56% POPC, 20% CHL, 11% POPI, 9% POPE, and 4% PSM). Finally, the resulting system was embedded into an orthorhombic box of explicit waters and Na⁺/Cl⁻ ions at a concentration of 150 mM. Molecular dynamics (MD) simulations were performed on the Frontera supercomputer at the Texas Advanced Supercomputing Center (TACC) using NAMD 2.14 and CHARMM36m al-atom additive force fields. Excluding initial minimization and equilibration, a total of 310 ns were collected for analysis. We note that, except for Cys²⁶¹ and Cys⁴⁹⁸ S-nitrosylation, the model described here is the same as the ACE2/RBD complex presented in Barros et al., Biophys. J. (2020).

Analysis of center of mass (COM) distance was performed with compute_center_of_mass function within MDtraj. COM for each ACE2 protomer was calculated taking into account the amino acid backbones of residues 18-742. The distance between COMs was evaluated at each frame along a 310 ns trajectory for both the WT ACE2/RBD complex by Barros and the model of the S-nitrosylated-ACE2/RBD complex presented here. As a reference, the distance between COMs from the cryo-EM structure (PDB: 6M17) was also calculated. The simulations were visually inspected using VMD.

Aminoadamantane and Nitro-Aminoadamantane Drugs

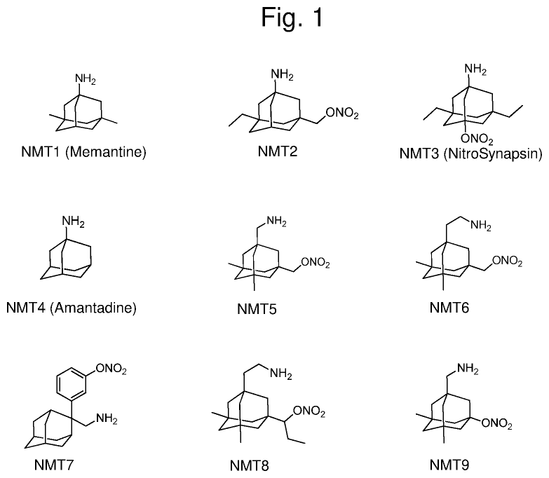

Nitro-aminoadamantane compounds (blindly coded NMT2, NMT3, and NMT5-NMT9) were synthesized, and have been described previously. The aminoadamantane compounds memantine and amantadine (blindly coded NMT1 and NMT4) were also tested in a masked fashion, and compound identities were not revealed until after experiments were completed and analyzed blindly. The structures of the compounds tested is provided in FIG. 1.

SARS-CoV-2 Virus Generation

Monkey Vero E6 cells (ATCC CRL-1586) were plated in a T225 flask with complete DMEM (Corning, 15-013-CV) containing 10% FBS, 1×PenStrep (Corning 20-002-CL), 2 mM L-glutamine (Corning, 25-005-CL) and incubated for overnight at 37° C. in a humified atmosphere of 5% CO₂. The medium in the flask was removed, and 2 ml of complete DMEM containing the WA1 strain of SARS-CoV-2 (USA-WA1/2020 [BEI Resources, NR-52281)) were added to the flask at a multiplicity of infection (MOI) of 0.5. After incubation for 30 minutes at 34° C. in a 5% CO₂ incubator, 30 ml of complete DMEM were added to the flask. The flask was then placed in a 34° C. incubator with 5% CO₂ for 5 days. On day 5 post infection, the supernatant was harvested and centrifuged at 1,000×g for 5 minutes. The supernatant was filtered through a 0.22 μM filter and stored at −80° C.

SARS-CoV-2/HeLa-ACE2 High-Content Imaging Assay for Infection

Control compounds solvated in DMSO were transferred into 384-well μclear-bottom plates (Greiner, Part. No. 781090-2B) using the ADS Labcyte Echo liquid handler. Aminoadamantane and nitro-aminoadamantane compounds to be screened were solvated in saline solution on ice immediately before use and transferred into the assay plates in 5 μl DMEM with 2% FBS (assay medium). HeLa-ACE2 cells were added to plates in assay medium at a density of 1.0×10³ cells per well to a 13 μl volume. Plated cells were transported to the BSL3 facility at Scripps Research, and within 1 h, 13 μl SARS-CoV-2 diluted in assay media was added at a multiplicity of infection (MOI)=0.65. Cells were incubated for 24 h at 34° C. in a 5% CO₂ incubator and then fixed with 8% formaldehyde for 1 h. Human polyclonal sera diluted at 1:500 in Perm/Wash buffer (BD Biosciences, 554723) were added to the plate and incubated at room temperature (RT) for 2 h. Six μg/ml of goat anti-human H+L conjugated Alexa 488 (Thermo Fisher Scientific, A11013) together with 8 μM of antifade-48-diamidino-2-phenylindole (DAPI; Thermo Fisher Scientific D1306) in SuperBlock T20 (PBS) buffer (Thermo Fisher Scientific, 37515) were added to the plate and incubated at RT for 1 h in the dark. Four fields were imaged per well using the ImageXpress Micro Confocal High-Content Imaging System (Molecular Devices) with a 10x objective. Images were analyzed using the Mufti-Wavelength Cell Scoring Application Module (MetaXpress) where DAPI staining was used to identify host-cell nuclei (the total number of cells in the images) and the SARS-CoV-2 immunofluorescence signal for identification of infected cells.

Uninfected Host Cell Cytotoxicity Counterscreen

Compounds were prepared and plated in 384-well plates as for the infection assay. HeLa-ACE2 cells were seeded in the assay-ready plates at 1.6×10³ cells/well in assay medium, and plates were incubated for 24 h at 37° C. with 5% CO₂. To assess cell viability, the Image-IT DEAD green reagent (Thermo Fisher, 110291) was used according to manufacturer's instructions. Cells were fixed with 4% paraformaldehyde (PFA) and counterstained with DAPI. Fixed cells were imaged using the ImageXpress Micro Confocal High-Content Imaging System (Molecular Devices) with a 10x objective, and total live cells per well quantified in the acquired images using the Live Dead Application Module (MetaXpress).

Data Analysis of the Compound Screening Results

The in vitro infection assay and the host cell cytotoxicity counterscreen data were uploaded to Genedata Screener, Version 16.0. Data were normalized to neutral (DMSO) minus inhibitor controls (2.5 μM remdesivir for antiviral effect, and 10 μM puromycin for infected host cell toxicity). For the uninfected host cell cytotoxicity counterscreen, 10 μM puromycin (Sigma) was used as a positive control. For dose-response experiments, compounds were tested in technical triplicates, and dose curves were fitted with the four parameter Hill Equation. Replicate data were analyzed using median condensing.

Pseudovirel Entry Assay

To measure SARS-CoV-2 viral infectivity, we performed pseudoviral entry assays as previously described. In brief, HEK293T cells were transiently co-transfected with MLV-gag/pol, MLV-CMV-Luciferase plasmid, and SARS-CoV-2 or VSV-G plasmid. Two days later, supernatants containing pseudotyped virus particles were collected. To assay for pseudoviral entry, HeLa-ACE2 cells were seeded in 96-well plates at 10,000 cells/well (PerkinElmer Inc. 6005680). One day later, cells were inoculated with diluted pseudovirus. After 48 h to allow for viral transduction, cells were lysed and assayed for luciferase activity by Steady-Glo® (Promega, E2510) according to the manufacturer's instructions. Luminescence was quantified using a Luminoskan Ascent plate leader (Thermo Fisher Scientific).

Pharmacokinetic Testing and Analysis

Golden Syrian hamsters (110-150 μm, n=3 for each compound) were dosed by oral gavage at 10 mg/kg. Blood samples were obtained at 30 min, 1, 3, 7, 24, 32, and 48 h. The blood samples were collected in BD Vacutainers containing sodium heparin, and centrifuged. The processed plasma samples are stored at −20° C. until high-performance liquid performance-tandem mass spectrometry (HPLC-MS/MS) analysis. Animals were then sacrificed, and lung and kidney tissues harvested for biotin-switch analysis for SNO-ACE2. To quantify the test compound in the collected plasma samples, a plasma calibration curve was generated by spiking aliquots of drug-free plasma with the test compound at the specified concentration levels. Spiked and collected plasma samples were treated with an aliquot of acetonitrile containing a known concentration of an Internal Standard (IS). The extraction solvent was analyzed on an Agilent 1100 LC mated to a AB Sciex 4000 Q TRAP MS. Separation of the analytes was achieved by using a Phenomenex Kinetex EVO C18 50×2.1 mm column (Phenomenex, 00B-4633-AN), and a mobile phase consisting of (A) 0.1% formic acid in water and (B) 0.1% formic acid in acetonitrile. The LC gradient consisted of a 0.5 mL/min flow rate starting at 100% of (A). The LC gradient then ramped to 90% (B) over 0.1 min, and held for 2.5 min. The gradient reverted to 100% (A) over 0.1 min and allowed for 2 min of re-equilibration time. Ionization spray (IS) voltage was set to 4000, with the source temp set at 400° C. Analytes were monitored by Multiple Reaction Monitoring (MRM) in positive ionization mode. Peak areas were recorded, and the concentrations of the test compound in the unknown plasma samples were quantified by the calibration curve using Sciex Analyst software (PE Sciex). Phoenix WinNonLin 8.1 software (Certara) was used in NCA mode to determine the PK parameters from the LC-MS/MS measured values.

Syrian Hamster COVID-19 Model

Eight-week old Golden Syrian hamsters (110-150 μm, Charles River) were administered via oral gavage either vehicle or the nitro-aminoadamantane candidate drugs NMT3 or NMT5 in a volume of 500 μl. Hamsters were then challenged with a dose of 1×10⁶ plaque forming units (PFU) of SARS-CoV-2 (USA-WA1/2020) by intranasal administration in a volume of 100 μl DMEM. At 2-d post infection, lung tissue was collected for assessing viral titers and histology as described.

Plaque Assay for SARS-CoV-2 Viral Titers

SARS-CoV2 titers were measured by homogenizing hamster lungs in DMEM 2% FBS using 100 μm cell strainers (Myriad 2825-8367). Homogenized lungs were titrated 1:10 over 6 steps and layered over Vero cells. After 1 h of incubation at 37° C., a 1% methylcellulose in DMEM overlay was added, and the cells were incubated for 3 days at 37° C. Cells were then fixed with 4% PFA and plaques were counted by crystal violet staining.

Lung Histology

Lung tissue from Golden Syrian Hamsters was stored in zinc formalin for 72 h. The tissue was processed for paraffin embedding, and sections cut at a thickness of 5 μm. The tissue was then stained with hematoxylin and eosin (H&E). The slides were scanned at 20X using an Aperio AT2 whole slide scanner.

Patch-Clamp Analysis of SARS-CoV-2 Envelope (E) Protein Viroporin Ion Channel Activity

Using the patch-clamp technique, we recorded whole-cell currents from untransfected HEK293T cells and cells transfected with SARS-CoV-2 envelope (E) protein. Cells were recorded at RT in Hepes-supplemented Hanks' balanced salt external solution with the following composition (in mM): NaCl 138, KCl 5.3, KH₂PO₄ 0.4, Na₂HPO₄ 0.3, NaHCO₃4.2, MgCl₂ 0.5, MgSO₄ 0.4, CaCl₂ 2.0, D-glucose 5.6 and Hepes 10; pH 7.4, 300 mOsm. Patch pipettes were filled with an intracellular pipette solution composed of (in mM): 113 Kgluconate, 6 KCl, 4.6 MgCl₂, 1.1 CaCl₂), 10 Hepes, 10 EGTA, 4 Na₂-ATP, 0.4 Na₂-GTP, pH adjusted to 7.3 with KOH; osmolality 291 mOsm. Whole-cell currents were recorded using a computer-controlled patch-damp amplifier (Multiclamp 700B, Molecular Devices). Traces were filtered at 2 kHz and sampled at 20 kHz. Drugs were dissolved in water and stored as 100 mM stocks at −20° C. During experiments, drugs were dissolved in extracellular solution and tested at concentrations of 5 and 10 μM. HEK293T cells were plated on 12-mm diameter glass coverslips coated with a mixture of rat tail type I collagen and poly-D-lysine. For transient expression in HEK293T cells, we used a transfection reagent (Fugene® HD, Promega) to co-transfect plasmids containing cDNAs for SARS-CoV-2 E protein (pGBW-m4133502, Addgene) and green fluorescent protein (GFP) at a ratio of 1:0.1 (0.5:0.05 μg/well, respectively).

Quantification and Statistical Analysis

A Power Analysis of our prior data was used to determine the number of replicates needed for statistical purposes. Data are expressed as mean±s.e.m. Differences between experimental groups were evaluated using an ANOVA followed by a post hoc Tukey's or Fisher's LSD test for multiple comparisons or a Student's t test for comparison of two groups. A P value<0.05 was considered significant. Statistical analyses were performed using GraphPad Prism software.

Results:

S-Nitrosylation of ACE2 at Cysteine 498 Destabilizes the ACE2 Dimer Interface to Affect SARS-CoV-2 Spike Protein Binding

Human ACE2 protein contains eight cysteine residues, six of which participate in formation of three pairs of disulfide bonds, and the remaining two (Cys²⁶¹ and Cys⁴⁹⁸) are present as free thiols (or thiolates) and thus potentially available for S-nitrosylation via reversible nucleophilic attack on a nitroso nitrogen to form an SNO-protein adduct. Accordingly, we performed site-directed mutagenesis of these cysteine residues in ACE2 and found that C261A, C498A, or C261A/C498A mutation significantly inhibited SNOC-mediated S-nitrosylation on biotin-switch assays, consistent with the notion that these two cysteine residues are targets of S-nitrosylation. Moreover, mass spectrometry confirmed the presence of S-nitrosylated ACE2 at Cys²⁶¹ and Cys⁴⁹⁶ after exposure to SNOC.

Notably, these S-nitrosylation sites are located near the collectrin-like domain (CLD) region rather than the spike protein-binding domain region of ACE2. This suggests that S-nitrosylation may affect the conformation of ACE2 protein at some distance from the S-nitrosylated cysteine residue(s) to diminish binding of ACE2 to trimeric S protein. Accordingly, explicitly solvated, all-atom molecular dynamics simulations of the S-nitrosylated-ACE2/RBD complex in plasma membrane show that the distance between each S-nitrosylated ACE2 protomer's center of mass is overall much longer and more broadly distributed than in the simulations of wild-type (WT) ACE2 dimer. This behavior indicates a certain extent of destabilization of the dimer interface imparted by S-nitrosylation, particularly of C⁴⁹⁸. Specifically, at the beginning of the simulations, the S-nitrosylated-ACE2/RBD model displays a hydrogen bond between Q175_{A} and Q139_{B}, which is then interchanged with D136. This is the only interaction between the peptidase domains (PD) of the two protomers, as also reported for the initial cryo-EM structure. Notably, over the course of our simulations, this interaction is progressively lost, leading to a partial disruption of the PD dimeric interface and transient detachment of the two protomers. Therefore, we hypothesize that the addition of S-nitrosylation at the side chain of C⁴⁹⁸, which is located in the vicinity of Q¹⁷⁵, could be sufficient to induce rearrangement in the packing of secondary structural elements of this region, leading in turn to the disruption of the only point of contact between the two PDs of ACE2. The loss of this contact may potentially trigger a further destabilization at the level of the dimeric interface between the neck domains. Alteration of ACE2 dimer stability has the potential to interfere with the SARS-CoV-2 spike binding, thus abrogating infection.

Screening Nitro-Aminoadamantane Compounds Against SARS-CoV-2 Infectivity

We examined the effect of nitro-aminoadamantane compounds on SARS-CoV-2 activity. Aminoadamantanes directly bind to and inhibit the activity of the ion channel formed by the envelope (E) protein channel of SARS-CoV-2. We screened our series of nitro-aminoadamantane compounds as potential therapeutic drugs against SARS-CoV-2—these drugs would be expected to both S-nitrosylate ACE2 to inhibit viral entry and block the viral channel—thus offering a dual mechanism of action. Specifically, we tested in a masked fashion the efficacy against live SARS-CoV-2 in HeLa-ACE2 cells of aminoadamantanes (memantine/blindly coded as NMT1 or amantadine/NMT4) and nitro-aminoadamantane compounds (NMT2, NMT3 and NMT5-NMT9). As positive controls, we used remdesivir, apilimod, and puromycin. In determining the therapeutic potential of these compounds, we considered the selectivity index (SI) that compares a compound's half-maximal non-specific cytotoxicity (CC₅₀) in the absence of infection to its half-maximal effective antiviral concentration (EC₅₀) (CC₅₀/EC₅₀) (see Table 1).

TABLE 1

| | Selectivity | CoV-2 | HeLa-ACE2 |
|---|---|---|---|
| | Index | EC50 | EC50 |
| Compound | CC50/EC50 | [uM] | [uM] |
| Apilimod - | >332.11 | 0.0072 | >2.40 |
| control | | | |
| Remdesivir - | >16.94 | 0.1417 | >2.40 |
| control | | | |
| Puromycin - | 0.728 | 0.6174 | 1.47 |
| control | | | |
| NMT6 | 5.32 | 3.3159 | 10.46 |
| NMT8 | 4.36 | 3.7544 | 8.37 |
| NMT5 | 9.21 | 5.2827 | 25.50 |
| NMT2 | 5.40 | 21.8839 | 121.85 |
| NMT1 | 2.69 | 53.8823 | 151.23 |
| (memantine) | | | |
| NMT4 | >2.86 | 70.0234 | >200 |
| (amantadine) | | | |
| NMT3 | >2.28 | 87.6790 | >200 |
| NMT9 | n/a | >200 | >200 |
| NMT7 | 1.88 | 90.6749 | >200 |

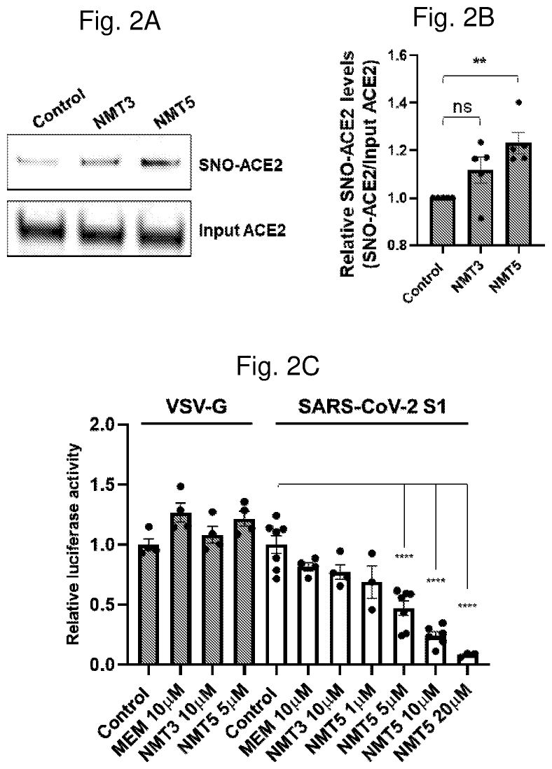

The SI can be considered an in vitro indicator of therapeutic index and ideally would approach 10. Among the 9 aminoadamantane or nitro-aminoadamantane compounds tested. NMT5 displayed the best combination of EC₅₀ and CC₅₀ (SI=9.2) with an EC₅₀ for protection against SARS-CoV-2 of 5.28 μM. This concentration of compound is well within the micromolar amounts attainable in human tissues at well-tolerated doses of aminoadamantane and nitro-aminoadamantane compounds that have been tested. Additionally, NMT3 (also known as NitroSynapsin), which was already being developed for CNS indications displayed some degree of protection against SARS-CoV-2 with an EC₅₀ of 87.7 μM, although this value may be artificially high due to the short half-life of NMT3 in aqueous solution under in vitro conditions. Hence, these two compounds were advanced for further study. We next asked if NMT3 and NMT5 could S-nitrosylate ACE2. We found that NMT5>NMT3 effectively S-nitrosylated ACE2 both in vitro in HeLa-ACE2 cells and in vivo in Syrian hamsters, as assessed by the biotin-switch assay (see FIGS. 2A and 2B). Notably, a statistically significant increase in the level of S-nitrosylated ACE2 was observed in the SARS-CoV-2 target tissues of lung and kidney at 48 h after oral administration of a single dose of drug at 10 mg/kg.

Compound NMT5 S-Nitrosylates ACE2 and Blocks SARS-CoV-2 Viral Entry

Since we had found that S-nitrosylation of ACE2 inhibited the binding of SARS-CoV-2 spike protein, we next asked if NMT3—or NMT5-mediated SNO-ACE2 formation could prevent viral entry into host cells. To test this premise, we employed a replication-deficient murine leukemia virus (MLV)-based SARS-CoV-2 spike protein pseudotype virus. We examined whether NMT3 and NMT5 could suppress infection with this SARS-CoV-2 pseudovirus. We found that NMT5 inhibited SARS-CoV-2 pseudoviral entry in a dose-dependent manner, with 5 μM inhibiting 53%, 10 μM 76%, and 20 μM 92% (see FIG. 2C). NMT3 showed more limited ability to suppress pseudovirus entry, ˜24% at 10 μM. In contrast, the pseudovirus entry assay performed with control vesicular stomatitis virus G protein (VSV-G) was unaffected by NMT3 or NMT5 (see FIG. 2C). These results are consistent with the notion that NMT5>>NMT3-mediated S-nitrosylation of ACE2 can inhibit SARS-CoV-2 entry into host cells.

Next, we sought to determine if NMT5 could modify ACE2 at both of the cysteine residues (Cys²⁶¹ and Cys⁴⁹⁸) that we demonstrated to be susceptible to S-nitrosylation by SNOC. Analysis by cysteine mutation revealed that NMT5 preferentially S-nitrosylated Cys⁴⁹⁸ over Cys²⁶¹. Interestingly, the crystal structure of ACE2 shows that an acid/base motif (comprised of Glu⁴⁹⁵ and Asp⁴⁹⁹), which under some conditions may facilitate S-nitrosylation, is present near Cys⁴⁹⁸, while only a partial motif (represented by Asp⁶⁰⁹) is found near Cys²⁶¹. This observation is consistent with prior findings that potent or supraphysiological amounts of NO donors such as SNOC can S-nitrosylate cysteine residues surrounded by no motif or only a partial SNO motif, whereas a full SNO motif can facilitate S-nitrosylation by less potent donors, presumably like NMT5. Mechanistically, cysteine thiol groups (in fact, thiolates) are nucleophiles that can perform reversible nucleophilic attack on an electrophilic nitroso nitrogen. The local environment of the thiolate anion can kinetically favor one electrophile over another; moreover, the bulky R-group of NMT5, as an RNO donor compared to the small molecule SNOC, could sterically hinder reactivity. Notably, concentrations of NMT5 that significantly inhibited viral entry (˜10 μM) failed to S-nitrosylate TMPRSS2, demonstrating relative selectivity of NMT5 for ACE2 at the cell surface.

To further investigate the effect of NMT5 on SARS-CoV-2 spike protein binding to ACE2, we performed co-immunoprecipitation (co-IP) experiments of these two proteins in the presence and absence of NMT5 using anti-ACE2 antibody for IP. As expected, the two proteins co-IP'd, as evidenced on immunoblots. NMT5 (5 μM) significantly diminished this co-IP, consistent with the notion that the drug inhibited the binding of spike protein to ACE2. As controls, the spike protein was not co-IP'd with cysteine mutant ACE2(C498A) or with double mutant ACE2(C261A/C498A), although mutant ACE2(C261A) was still co-IP'd. These data are consistent with the notion that S-nitrosylation predominantly of C⁴⁹⁸ of ACE2 is important for spike protein binding to ACE2. Moreover, NMT5 inhibited co-IP of the spike protein and ACE2(C261A), while having no effect on mutant ACE2(C498A) or ACE2(C261A/C498A) binding. Taken together, these results are consistent with the notion that NMT5 inhibits SARS-CoV-2 spike protein from binding to ACE2 and thus virus entry into the cell via S-nitrosylation of ACE2.

NMT5 Blocks SARS-CoV-2 E Protein Ion Channel Activity

To investigate further the effects of the aminoadamantane moiety on SARS-CoV-2, we assessed the ability of the aminoadamantane compound, memantine, and the nitro-aminoadamantane lead candidate, NMT5, to block ion channel activity of the envelope (E) protein of SARS-CoV-2 using the patch-clamp technique. For this purpose, we transiently transfected HEK293T cells with the viroporin channel and assessed voltage-dependent currents (vs. uninfected cells) in the presence and absence of drug. Under our conditions, we found that the presence of the E protein resulted in a robust voltage-dependent current carried by K⁺ that was inhibited in a dose-dependent manner by memantine and with greater potency by NMT5. Notably, the low micromolar concentrations needed to see these effects are within attainable levels in mammalian plasma and tissues, as shown in pharmacokinetic (PK) studies, and have proven to be safe in animal toxicity studies.

NMT5 Decreases SARS-CoV-2 Infection In Vivo in the Syrian Hamster Model

In preparation for in vivo drug candidate efficacy testing in a COVID-19 small animal model, we next performed 48-h PK studies after a single oral dose of NMT3 or NMT5 at 10 mg/kg in −150 μm Syrian hamsters. We found a half-life in plasma for NMT3 of ˜2 h and for NMT5 of 10.6 h. The mean Cₘₐₓ for NMT5 was ˜0.2 μM and ˜0.12 μM for NMT3. NMT5 was found to be far more stable than NMT3 by mass spectrometry analysis. We then tested the effect of a single oral loading dose of the nitro-aminoadamantanes in vivo on viral titers and hence infectivity in the Syrian hamster model of COVID-19. We performed a dose-escalation trial, with doses from 10 to 200 mg/kg of NMT3 or NMT5. Based on the PK results, at the highest dose, the plasma levels should approach the EC₅₀ found in our in vitro screens to prevent viral infectivity, at least for the more stable drug NMT5. We found in the Syrian hamster model that a single dose of 200 mg/kg of NMT5 but not NMT3 statistically decreased live viral titers of SARS-CoV-2 at 48 h post infection by approximately two orders of magnitude, as measured by plaque assay. Additionally, histological examination revealed less COVID-19-related hemorrhage in the lungs of NMT5-treated hamsters compared to vehicle at 48 h.

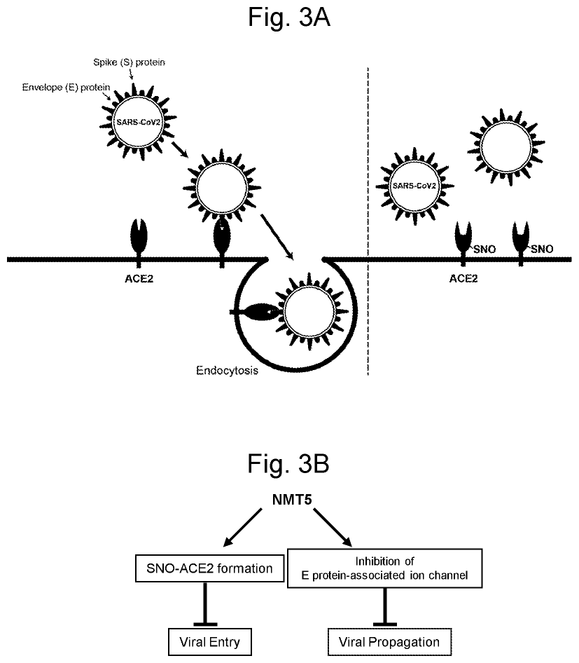

Collectively, these findings show that ACE2 can be S-nitrosylated to inhibit binding of SARS-CoV-2 spike protein, thus inhibiting viral entry, infectivity, and cytotoxicity. Taking advantage of this finding, the novel nitro-aminoadamantane compound, NMT5, provides a dual mechanism of inhibition of SARS-CoV-2 activity: S-nitrosylating ACE2 via the nitrate group to inhibit infectivity, plus inhibiting viroporin E protein channel activity via the aminoadamantane moiety, which is required for viral propagation (see FIGS. 3A and 3B). These mechanistic insights should facilitate future optimization of such nitro-aminoadamantane drugs for antiviral therapy for COVID-19.

### OTHER EMBODIMENTS

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims. Other embodiments are within the claims.

What is claimed is:

1. A method of treating a betacoronavirus infection in a human subject, said method comprising administering to the subject a therapeutically effective amount of a nitro-aminoadamantane compound or a pharmaceutically acceptable salt thereof.

2. A method of ameliorating one or more symptoms of a betacoronavirus infection in a human subject, said method comprising administering to the subject a therapeutically effective amount of a nitro-aminoadamantane compound or a pharmaceutically acceptable salt thereof.

3. A method of inhibiting the progression of a betacoronavirus infection in a human subject, said method comprising administering to the subject a therapeutically effective amount of a nitro-aminoadamantane compound or a pharmaceutically acceptable salt thereof.

4. A method of reducing the likelihood of betacoronavirus infection in a human subject at risk thereof, said method comprising administering to the subject a therapeutically effective amount of a nitro-aminoadamantane compound or a pharmaceutically acceptable salt thereof.

5. The method of any one of claims 1-4, wherein the risk of pneumonia or pneumonitis in the subject is reduced.

6. The method of any one of claims 1-4, wherein the risk of hospitalization or the duration of hospitalization of the subject is reduced.

7. The method of any one of claims 1-4, wherein the risk of acute respiratory distress syndrome in the subject is reduced.

8. The method of any one of claims 1-4, wherein the risk of respiratory failure in the subject is reduced.

9. The method of any one of claims 1-4, wherein the risk of septic shock in the subject is reduced.

10. The method of any one of claims 1-4, wherein the risk of organ failure in the subject is reduced.

11. The method of any one of claims 1-4, wherein the risk of death in the subject is reduced.

12. The method of any one of claims 1-4, wherein the risk of cytokine storm in the subject is reduced.

13. The method of any one of claims 1-12, wherein the administering occurs between once per week to three times per day.

14. The method of any one of claims 1-12, wherein the administering occurs once per day.

15. The method of any one of claims 1-12, wherein the administering is twice per day.

16. The method of any one of claims 1-15, wherein the administering occurs over a treatment period.

17. The method of claim 16, wherein the treatment period is about 1 day to about 21 days.

18. The method of claim 16, wherein the treatment period is about 1 week to about 6 weeks.

19. The method of any one of claims 1-18, wherein the nitro-aminoadamantane compound is administered orally.

20. The method of any one of claims 1-19, wherein the subject is being hospitalized for the betacoronavirus infection.

21. The method of any one of claims 1-20, wherein the subject has a pre-existing condition that places the subject at higher risk of pneumonitis, pneumonia, acute respiratory distress syndrome, respiratory failure, septic shock, organ failure, cytokine storm, or death.

22. The method of claim 21, wherein the pre-existing condition is selected from cardiovascular disease, diabetes, chronic respiratory disease, hypertension, immune deficiency, and obesity.

23. The method of any one of claims 1-22, wherein the subject is at least 40 years old, at least 50 years old, at least 60 years old, at least 70 years old, or at least 80 years old.

24. The method of any one of claims 1-23, wherein the betacoronavirus is SARS-CoV-2.

25. The method of any one of claims 1-23, wherein the betacoronavirus is SARS-CoV-1.

26. The method of any one of claims 1-23, wherein the betacoronavirus is MERS-CoV.

27. The method of any one of claims 1-26, wherein the nitro-aminoadamantane compound is a compound of any one of formulas (I)-(V).

28. The method of any one of claims 1-26, wherein the nitro-aminoadamantane compound is selected from:

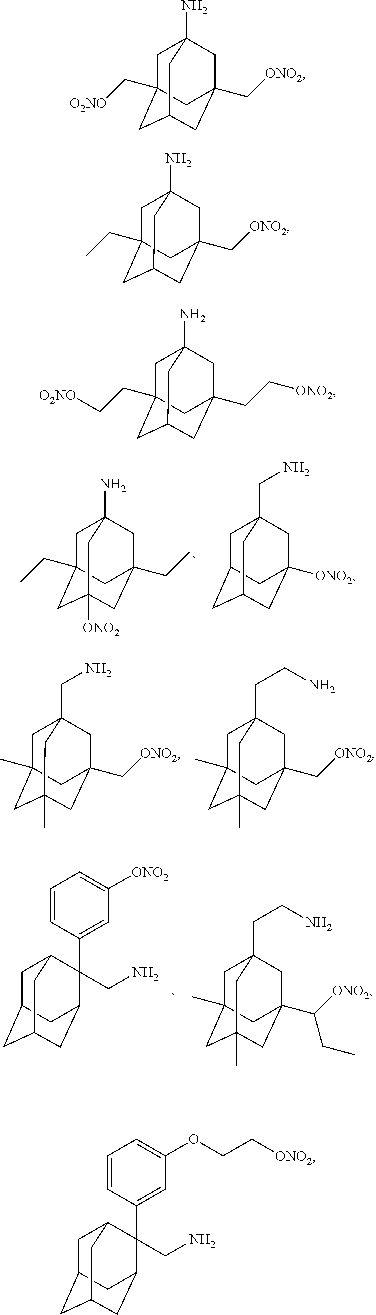

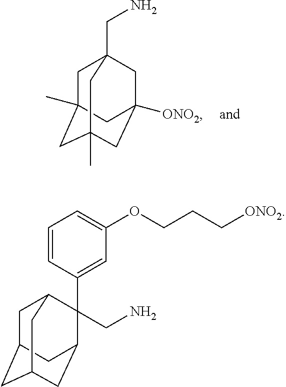

29. The method of claim 28, wherein the nitro-aminoadamantane compound is selected from: 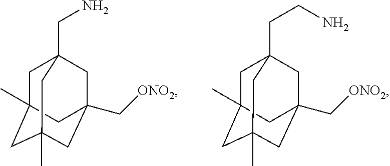
or a pharmaceutically acceptable salt thereof.

30. The method of claim 2, wherein the one or more symptoms of betacoronavirus infection comprises a reduction in mental clarity and/or inability to focus.
